(19) United States
(12) Patent Application Publication
(10) Pub. No.: US 2013/0136721 A1
(43) Pub. Date: May 30, 2013

(54) Compositions and Methods of Generating a Differentiated Mesodermal Cell

(71) Applicants: Elias Zambidis, Ellicott City, MD (US); Paul Burridge, Palo Alto, CA (US)

(72) Inventors: Elias Zambidis, Ellicott City, MD (US); Paul Burridge, Palo Alto, CA (US)

(73) Assignee: THE JOHNS HOPKINS UNIVERSITY, Baltimore, MD (US)

(21) Appl. No.: 13/577,944

(22) PCT Filed: Feb. 9, 2011
(86) PCT No.: PCT/US11/24142

§ 371 Date: Feb. 7, 2013

Related U.S. Application Data
(60) Provisional application No. 61/302,714, filed on Feb. 9, 2010.
(60) Provisional application No. 61/426,292, filed on Dec. 22, 2010.

(51) Int. Cl. C12N 5/071

(52) U.S. Cl. CPC C12N 5/0602
USPC 424/93.7; 435/366; 435/34; 435/305.1; 435/29; 435/32; 435/304.1

(57) ABSTRACT

The invention features a culture system, culture system components and culture methods that are useful for the rapid and reliable generation of differentiated mesodermal cells, including cardiac myocytes, from stem cells, such as human embryonic stem cells and human induced pluripotent stem cells differentiated mesodermal cells, including differentiated cardiac myocytes.

### CROSS-REFERENCE TO RELATED APPLICATION

This application claims the benefit of the following U.S. Provisional Application Nos. 61/302,714, filed Feb. 9, 2010, and 61/426,292, filed Dec. 22, 2010, the entire contents of which are incorporated herein by reference.

### STATEMENT OF RIGHTS TO INVENTIONS MADE UNDER FEDERALLY SPONSORED RESEARCH

This work was supported by the following grants from the National Institutes of Health, Grant Nos: K08 HL077595, RFA-MD-08-1 and RFA-MD-09-3. The government has certain rights in the invention.

### BACKGROUND OF THE INVENTION

Recent work has indicated that human pluripotent stem cells can be induced to differentiate into virtually any adult cell type. Such cells are useful in repairing or regenerating cells, tissues, or organs damaged due to undesirable cell death (e.g., cell death related to ischemic injury, degenerative conditions), trauma or congenital defects. However, existing methods for inducing differentiation into mesodermal cells, including cells of the cardiac lineage, are inadequate. Existing methods for generating cardiac cells and other differentiated mesodermal cell types typically result in poor yields and require long differentiation times. Differentiated cardiomyocytes are particularly useful in repairing or regenerating cardiac tissue that has sustained an ischemic damage or other injury. Given that heart disease affects one in three adults in the U.S., compositions and methods for quickly and reliably generating cells useful in the repair or regeneration of cardiac tissues are urgently required.

### SUMMARY OF THE INVENTION

As described below, the present invention features compositions and methods that induce the differentiation of mesodermal cells from pluripotent stem cells, and methods of using the differentiated mesodermal cells to replace or regenerate a cell, tissue or organ characterized by a deficiency in cell number or cell function.

In one aspect, the invention features a method for generating a differentiated mesodermal cell, the method involving culturing a human stem cell as a monolayer on a proteinaceous cell culture matrix that supports cell adhesion; promoting the aggregation of the cells by culturing the cells in media containing poly(vinyl alcohol), BMP4, and FGF2; maintaining the cells in culture for another forty-eight hours under conditions that support mesodermal lineage specification; and maintaining the cells for six additional days under conditions that promote human embryonic body maturation, thereby generating a differentiated mesodermal cell. In one embodiment, the method further involves plating cells at about 5000 cells per well of a 96-well plate in culture media containing 25 ng ml⁻¹ BMP4, 5 ng ml⁻¹ FGF2 and cell culture media containing at least 4% PVA. In another embodiment, the method further involves centrifuging the culture to force the cells to aggregate. In another embodiment, the method the conditions include culturing the cells in media containing FBS or a FBS-substitute and/or culturing the cells in media containing 4% PVA.

In another aspect, the invention features a method for generating a differentiated mesodermal cell, the method involving culturing a human stem cell or human induced pluripotent stem cell in medium conditioned on irradiated mouse embryonic fibroblasts seeded at 6×10⁴ cells/cm² for 22-26 hour, where the conditioned media contains at least about 4 ng/ml FGF2; culturing a human stem cell or human induced pluripotent stem cell as a monolayer on a proteinaceous cell culture matrix that supports cell adhesion; plating cells from at about 5000 cells per well of a 96-well plate in culture media containing 25 ng ml⁻¹ BMP4, 5 ng ml⁻¹ FGF2 and cell culture media containing at least 4% PVA and centrifuging the culture to force the cells to aggregate; maintaining the cells in culture for another forty-eight hours in media containing FBS or a FBS-substitute, 25 ng/ml BMP4 and 5 ng ml⁻¹ FGF2, thereby promoting mesodermal lineage specification; and maintaining the cells for six additional days in media containing 4% PVA or media containing FBS or an FBS-substitute thereby generating a differentiated mesodermal cell. In one embodiment, the medium is RPMI-BSA-PVA medium that contains or is RPMI with 2 mM L-glutamine, 4 mg/mL PVA, 1× Chemically Defined Lipid Concentrate (commercially available from Invitrogen), 400 mM 1-thioglycerol, 10 ug/mL insulin. In another embodiment, the method further involves detecting and isolating hEBs that beat.

In another aspect, the invention features a differentiated mesodermal cell generated according to the method of any previous aspect.

In another aspect, the invention features a culture system containing one or more containers of media, where a first cell culture media that promotes mesodermal lineage specification, the media containing FBS or a FBS-substitute, about 25 ng/ml BMP4 and about 5 ng ml⁻¹ FGF2; a second cell culture media that promotes cell aggregation containing about 25 ng ml⁻¹ BMP4, about 5 ng ml⁻¹ FGF2 and about 4% PVA; a third cell culture media for maintaining human embryonic stem cells or human induced pluripotent cells in culture, where the medium is conditioned on irradiated mouse embryonic fibroblasts seeded at about 6×10⁴ cells/cm² for about 22-26 hour, and where the conditioned media contains at least about 4 ng/ml FGF2; and directions for the use of the culture system to promote mesodermal cell proliferation according to any previous aspect. In one embodiment, the culture system further includes culture flasks or culture plates.

In yet another aspect, the invention features a method of ameliorating cell or tissue loss in a subject in need thereof, the method involving delivering to the subject an effective amount of a cell generated according to the method of any previous aspect. In one embodiment, the cell or tissue loss or damage is associated with a condition selected from the group consisting of myocardial infarction, heart failure, cardiomyopathy, congenital heart disease, nutritional diseases, ischemic or non-ischaemic cardiomyopathy, hypertensive cardiomyopathy, valvular cardiomyopathy, inflammatory cardiomyopathy, cardiomyopathy secondary to a systemic metabolic disease, alcoholic cardiomyopathy, diabetic cardiomyopathy, or restrictive cardiomyopathy.

In another aspect, the invention features a method of treating a damaged cardiac tissue in a subject in need thereof, the method involving delivering to the subject an effective amount of a cardiac myocyte generated according to the method of any previous aspect.

In another aspect, the invention features a pharmaceutical composition containing a differentiated mesodermal cell generated according to the method of any previous aspect in a pharmaceutically acceptable excipient.

In another aspect, the invention features a kit containing the culture system of a previous aspect and instructions for generating a differentiated mesodermal cell in accordance with any previous aspect.

In another aspect, the invention features a kit containing a differentiated mesodermal cell obtained according to any previous aspect, and instructions for engraftment of the differentiated mesodermal cell in a subject.

In another aspect, the invention provides a method for drug screening that involves contacting a cardiac cell delineated herein with an agent and detecting an alteration in the survival or biological activity of the cell. In one embodiment, the method detects an increase or decrease in cell death. In another embodiment, the method detects an increase or decrease in biological activity of the cell.

In various embodiments of any previous aspect or any other aspect of the invention delineated herein, the stem cell is any one or more of an induced pluripotent stem cell, human embryonic stem cell, mesodermal stem cell, and other mesodermal stem cell. In other embodiments, an induced pluripotent stem cell is derived from a somatic cell (e.g., keratinocyte, epidermal cell, fibroblast, and their progenitor cells). In other embodiments, the human embryonic stem cell or human induced pluripotent stem cell is maintained in a culture containing medium conditioned on irradiated mouse embryonic fibroblasts seeded at 6×10⁴ cells/cm² for about 22-26 hour. In other embodiments, the media for the culture of pluripotent cells as a monolayer is a commercially available media that is any one or more of mTeSR1, TeSR2 (Stem Cell Technologies), Nutristem (Stemgent), StemPro hESC SFM (Invitrogen) or media supplemented with Xeno-Free Knockout Serum Replacement and Xeno-Free Growth Factor Cocktail (Invitrogen), In other embodiments, the conditioned media further contains at least about 4 ng/ml FGF2. In other embodiments, the media of is RPMI-PVA medium that is RPMI (with 2 mM L-glutamine), between 1-10 mg/ml PVA (e.g., 1, 3, 4, 5, 10 mg/mL PVA), 1× Lipid Concentrate, 400 mM 1-thioglycerol, and 10 ug/mL Insulin. In other embodiments, the method further involves identifying an differentiated mesodermal phenotype by detecting an increase in a mesodermal marker, mesodermal morphology, or mesodermal function that is not detectably expressed or expressed only nominally in a corresponding control cell. In other embodiments, the differentiated mesodermal cell expresses one or more mesodermal markers selected from the group consisting of T, MIXL1, GSC, EOMES and MESP1. In other embodiments, the method generates about 75%, 85%, 90% or 85% differentiated mesodermal cells after about 9 days.

The invention provides positions and methods that induce the differentiation of mesodermal cells from pluripotent stem cells. Compositions and articles defined by the invention were isolated or otherwise manufactured in connection with the examples provided below. Other features and advantages of the invention will be apparent from the detailed description, and from the claims.

### DEFINITIONS

By “differentiated mesodermal cell” is meant a cell that expresses mesodermal markers. Mesodermal markers include, but are not limited to T (Brachury) and MIXL1.

By “cardiac myocyte” is meant a cell expressing cardiac markers and/or having cardiac myocyte function. Cardiac markers include, but are not limited to NKX2-5, TNNT2 and MYH6.

By “alteration” is meant a change (increase or decrease) in the expression levels of a gene or polypeptide as detected by standard art known methods such as those described above. As used herein, an alteration includes a 10% change in expression levels, preferably a 25% change, more preferably a 40% change, and most preferably a 50% or greater change in expression levels.

By “autologous” is meant cells from the same subject.

By “compound” is meant any small molecule chemical compound, antibody, nucleic acid molecule, or polypeptide, or fragments thereof.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

The term “engraft” as used herein refers to the process of cell incorporation into a tissue of interest in vivo through contact with existing cells of the tissue.

By “exogenously expressed” is meant expressing a polypeptide or polynucleotide that is not naturally expressed at a functionally significant level in the cell. For example, a recombinant polypeptide that is introduced into the cell using an expression vector is an example of an exogenously expressed polypeptide. In other example, the cell expresses a heterologous polypeptide or polynucleotide.

A “labeled nucleic acid or polypeptide” is one that is bound, either covalently, through a linker or a chemical bond, or noncovalently, through ionic bonds, van der Waals forces, electrostatic attractions, hydrophobic interactions, or hydrogen bonds, to a label such that the presence of the nucleic acid or probe may be detected by detecting the presence of the label bound to the nucleic acid or probe.

By “increases or decreases” is meant a positive or negative alteration. Such alterations are by 5%, 10%, 25%, 50%, 75%, 85%, 90% or even by 100% of a reference value.

By “induced pluripotent stem cell” is meant a differentiated somatic cell that acquires pluripotency by the exogenous expression of one or more transcription factors in the cell.

By “isolated” is meant a material that is free to varying degrees from components which normally accompany it as found in its native state. “Isolate” denotes a degree of separation from original source or surroundings.

By “isolated nucleic acid molecule” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule which is transcribed from a DNA molecule, as well as a recombinant DNA which is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. In one embodiment, the preparation is at least 75%, 85%, 90%, 95%, or at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “marker” is meant any protein or polynucleotide having an alteration in expression level or activity that is associated with a disease or disorder.

By “matrix” is meant a medium that provides for the survival, proliferation, or growth of one or more cells. In one embodiment, a matrix is a cell scaffold comprising a biodegradable medium.

By “naturally occurs” is meant is endogenously expressed in a cell of an organism.

By “obtaining” as in “obtaining the polypeptide” is meant synthesizing, purchasing, or otherwise acquiring the polypeptide.

By “polypeptide” is meant any chain of amino acids, regardless of length or post-translational modification.

By “positioned for expression” is meant that the polynucleotide of the invention (e.g., a DNA molecule) is positioned adjacent to a DNA sequence that directs transcription and translation of the sequence (i.e., facilitates the production of, for example, a recombinant polypeptide of the invention, or an RNA molecule).

By “promoter” is meant a polynucleotide sufficient to direct transcription. Exemplary promoters include nucleic acid sequences of lengths 100, 250, 300, 400, 500, 750, 900, 1000, 1250, and 1500 nucleotides that are upstream (e.g., immediately upstream) of the translation start site.

The term “self renewal” as used herein refers to the process by which a stem cell divides to generate one (asymmetric division) or two (symmetric division) daughter cells with development potentials that are indistinguishable from those of the mother cell. Self renewal involves both proliferation and the maintenance of an undifferentiated state.

The term “stem cell” is meant a pluripotent cell or multipotent stem cell having the capacity to self-renew and to differentiate into multiple cell lineages.

By “stem cell generation” is meant any biological process that gives rise to stem cells. Such processes include the differentiation or proliferation of a stem cell progenitor or stem cell self-renewal.

By “stem cell progenitor” is meant a cell that gives rise to stem cells.

By “subject” is meant a mammal, including, but not limited to, a human or non-human mammal, such as a bovine, equine, canine, ovine, or feline.

By “syngeneic,” as used herein, refers to cells of a different subject that are genetically identical to the cell in comparison.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

By “reference” is meant a standard or control condition.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

Any compositions or methods provided herein can be combined with one or more of any of the other compositions and methods provided herein.

### BRIEF DESCRIPTION OF THE DRAWINGS

FIGS. 1A and 1B are schematic diagrams showing the development of a strategy for the optimization of cardiac differentiation. FIG. 1A shows a schematic of an early cardiac differentiation strategy. FIG. 1B shows that pilot experiments allowed development of a prototype system in which BMP4 was used from d0-d4 and removed from the mass culture step which eliminates the inter-hEB paracrine effect and prevents hEB from adhering to each other. This method used a modified CDM-PVA (replacing insulin and transferrin with 1×ITS-X (Insulin-Transferrin-Selenium-x). FIG. 1C shows a final four step optimized differentiation strategy detailing the use of hESC/hIPSC passaged one day prior to aggregation, 5,000 cells in RPMI+PVA media for 2 days followed by 2 days in RPMI+FBS and finally adherence in RPMI+PVA.

FIGS. 2A-2C show physical factor and media formulation strategy. FIG. 2A provides a schematic representation of the variables considered in optimizing the differentiation system. FIG. 2B provides a table of methodology used for rounds of optimization of each of the phases of differentiation. FIG. 2C shows a heat-map of optimized media formulations and physical factors for cardiac differentiation of H9 hESC and provides a condensed schematic of the optimal cardiac differentiation media formulations and physical factors used in the optimized protocol. The midrange shading shown represents greater than 90% of hEB contracting on d9, light shading represents 50-90% of hEB contracting on d9, darkest shading represents less than 50% of hEB contracting on d9, and white represents 0 contracting hEB on d9. *BMP4 concentration requirements for hESC and hiPSC differ.

FIGS. 3A-3D show the optimization of cardiac differentiation of human pluripotent stem cells to near total efficiency. FIG. 3A is a schematic of the optimized cardiac differentiation system demonstrating: Phase 1, uniform growth of hESC/hiPSC as monolayers. Phase 2 (d0-d2), forced aggregation of 5000 single cell hESC or hiPSC in chemically defined RPMI+PVA medium consisting of RPMI, PVA, insulin, 1-thioglycerol, BMP4, FGF2 and Y-27632 in V96 plates. Phase 3 (d2-d4), cardiac specification using 20% FBS or hSA in RPMI 1640 with 1-thioglycerol. Phase 4 (d4+), cardiac development, hEB are allowed to adhere to U96 tissue culture treated plates in RPMI+PVA. FIGS. 3B and 3C are micrographs. FIG. 3B shows typical d2 human embryoid bodies (hEB) formed using the forced aggregation procedure in RPMI-PVA to demonstrate homogeneity in hEB size. Scale bar=500 μm. FIG. 3C shows typical d9 contracting hEB formed using the optimized cardiac differentiation method with the contracting area circled. In contrast to previous methods this system consistently and reproducibly produced hEB in which the entire hEB contracted. Scale bar=200 μm. FIG. 3D is a graph showing the efficiency of generation of contracting hEB produced in serum-containing (diamond, New system) vs. xeno- and serum-free conditions (box, Serum-free), with comparisons to a previous method (Δ, Previous system) and previously published methods (x, Typical) using suspension of colonies to form hEB in 20% FBS (new system n=48, serum-free n=5, previous system n=7, typical n=9. Error bars, ±s.e.m.

FIGS. 4A-4D show the controlled and reproducible growth of human pluripotent stem cells (hPSC) for subsequent cardiac differentiation. FIG. 4A is a schematic of monolayer hESC/hiPSC culture technique. This monolayer technique uses conditioned medium prepared in a defined manner, single-cell passaging, automated cell counting, plating cells at a known density and passaging every three days. During development the coating matrix Geltrex (Invitrogen), an alternative to Matrigel (BD Biosciences), was found to be suitable for use at a 1:400 dilution, greatly reducing costs of this method. Each cell line was plated at 1.25×106 cells per T25 flask and grown to 4−5×106 in three days. FIG. 4B is a graph showing the stable growth rate of H9 hESC (H9), hiPSC lines iPS(IMR90)-1 and iPS(IMR90)-4, 6.2, 6.11, 6.13. FIG. 4C provides two micrographs showing that the homogenous phenotype seen when culturing H9 hESC as feeder-free monolayers (left) in comparison to cells cultured as colonies on MEF (right). FIG. 4D is a FACS analysis showing higher SSEA4 and TRA-1-60 expression in monolayer cultures (left) than colonies on MEF (right)

FIG. 5 is a table showing the optimization of day 0-2 media formulation and growth factor variables. Optimal conditions for the d0-d2 phase 2 stage of differentiation were: (a) 25 ng mL⁻¹ of BMP4. (b) 5 ng ml⁻¹ FGF2. (c) RPMI. (e) 400 μM 1-thioglycerol. (f) 10 μg mL⁻¹ insulin and (k) 4 mg mL⁻¹ PVA, (i) 1× lipids and (I) 1 μM Y-27632. The addition of (d) L-glutamine, (g) transferrin, (h) L-ascorbic acid, (j) non-essential amino acids, or (k) the replacement of PVA with BSA either did not further enhance differentiation or had negative effects on differentiation

FIG. 6 is a table showing the optimization of forced aggregation hEB formation physical factors. (a) Forced aggregation input cell number per well between 500-20,000 cells, 3,000-10,000 cells was suitable for successful cardiac differentiation. hEB did not form from 500 or 1,000 cells. (b) Both V-bottom and U-bottom plates were successful for hEB formation, V-bottom plates were chosen due to the comparative ease of media change and prevention of loss of hEB. (c) Only day 2 was suitable for change of media RPMI-FBS. (d) hEB that were transferred to adherent plates before d4 quickly lost their structure. (e) U-bottom plates were chosen over F-bottom plates as hEB in the middle of the well were quicker to count. (f) Once Y-27632 was added to the media it was found that g-force was no longer required to induce aggregation. (g) The density at which the T25 flasks of pluripotent cells were split to the day before forced aggregation did not affect subsequent differentiation. (h) Passaging cells one day prior to forced aggregation rather than allowing them to grow to confluence was found to be crucial for efficient differentiation.

FIG. 7 is a table showing the optimization of day 2-4 media factors. (a) Only 20% FBS was suitable for >90% contracting hEB. (b) Manufacturer of FBS did not affect cardiac induction and human serum was as effective as FBS. (c) The addition of BMP4 did not enhance cardiac differentiation. (d) The addition of FGF2 also did not enhance cardiac differentiation. (e) As with d0-d2, only the basal medium RPMI was suitable for efficient cardiac differentiation. (f) Additional L-glutamine did not enhance cardiac differentiation. (g) 1-thioglycerol was the most suitable antioxidant for this phase. (h) Any level of supplementation with insulin during this phase completely ablated cardiac differentiation. (I, j, k, l) Neither transferrin, L-ascorbic acid, lipids or NEAA enhanced this phase of differentiation.

FIG. 8 is a table showing the optimization of day 4 onwards media formulation. (a) FBS, PVA or HSA was not required for the d4 onwards phase. (b) In contrast to the d2-d4 stage, insulin did not effect this d4+ phase. (c) transferrin was not required. (d) Supplemental lipids were also not required. (e) 1-thioglycerol was essential for this d4+ phase.

FIGS. 9A-9C are tables showing the elimination of interline variability of cardiac differentiation with PVA supplementation and physiological oxygen. FIG. 9A shows that increasing the concentration of PVA from 1 mg mL-1 to 4 mg mL-1 PVA in the d0-d2 media formulation enhanced the differentiation of iPS(IMR90)-1 and 6.2 hiPSC whilst not affecting H9 hESC (n=3). FIG. 9B shows that exposure of iPS(IMR90)-1 hEB to physiological (5%) O2 tensions from differentiation d0-d2 enhanced cardiac differentiation (n=3, p<0.005). Identical conditions did not affect H9 hESC differentiation (data not shown). FIG. 9C shows that combining physiological oxygen tension and 4 mg mL-1 PVA between d0-d2 eliminates interline variability in hiPSC differentiation (n=3). Error bars, ±s.e.m.

FIGS. 10A and 10B show the characterization of H9 hESC-derived cardiomyocytes. FIG. 10A is a table showing a comparison of Real Time quantitative RT-PCR for markers of pluripotency, mesoderm, cardiac progenitors, and cardiomyocytes during hESC differentiation using either the Previous system (diamonds) or New system (boxes). Analysis was performed using the ^{ΔΔ}Ct method with d0 as baseline and 18S as the control. FIG. 10B provides fluorescent micrographs showing that hESC-derived cardiomyocytes display striated ultra-structural expression of troponin I (green) and a-actinin (red) phenotype.

FIGS. 11A-11C show a quantitative assessment cardiomyocytes within contracting hEB. FIG. 11A provides micrographs showing that whole d9 H9 (left) and iPS(IMR90)-1 (right) hEB were stained with the cardiomyocyte specific mitochondrial dye TMRM. FIG. 11B shows the results of intracytoplasmic flow cytometry analysis of d9 H9 hEB: unstained control; hEB differentiated without BMP4 to prevent cardiomyocyte formation; hEB differentiated with BMP4 from d0-d2 demonstrating that hEB consisted of 63.6% cardiomyocytes. FIG. 11C shows the results of flow cytometry for troponin I (TNNI3).

FIGS. 12 A-12D show a demonstration of highly reproducible electrophysiological properties of contracting hEB by optical mapping. FIG. 12A-a shows the results of voltage micromapping. At far left is a phase contrast image of H9 hEB at 4× magnification. FIG. 12A-b shows a voltage activation map (arrows indicate direction of electrical wave propagating across hEB). FIG. 12A-c shows an Action potential duration (APD) map. FIG. 12A-d shows a representative transmembrane potential (Vm) trace at position denoted by the small square in a and b. FIG. 12B shows a mean APD and conduction velocity (CV) measurements from 19 hEB (error bars represent ±s.d.). Coefficient of variation (COV, population s.d. divided by mean) for APD was 0.30 and for CV was 0.88 across hEB population. COV within an individual hEB was calculated from multiple APD measurements across all of the recording sites for that hEB (panel (a) c) and was 0.042±0.030 (s.d.) when averaged across 19 hEB. FIGS. 12C-a and b show that the beta-adrenergic agonist isoproterenol shortened the mean APD in all 4 hEB by an average of 23±8 ms (mean±s.d.). * indicates p≈0.01 in a paired Student's t-test. FIG. 12D-a shows a time series of voltage maps demonstrates electrical coupling in an hESC-derived cardiomyocyte monolayer during 0.67 Hz pacing (pulse symbol indicates stimulus site, arrows indicate direction of propagation). A second, spontaneous activation site can be seen on the upper right at 40 ms. FIG. 12D-b shows a representative Vm traces, time aligned by the stimulus timing, taken at site x in a. Isoproterenol and pinacidil shortened the action potential (363±137 ms control (n=73 recording sites) vs. 257±56 ms pinacidil (n=64) vs. 262±107 ms isoproterenol (n=94), mean±s.d.).

FIGS. 13A-13C show optical mapping demonstrating electrophysiological function. FIG. 13A-a Intracellular calcium micromapping. FIG. 13A-a, Phase map of hEB at 6× magnification. FIG. 13A-b, Calcium map (arrows indicate direction of propagating calcium wave). FIG. 13A-c, Representative intracellular calcium (Cai) trace at position denoted by the box in a and b. FIG. 13B shows electrical coupling during voltage micromapping. FIG. 13B-a phase map of two hEB in close contact at 6×. FIG. 13B-b shows a time series of voltage maps demonstrates electrical coupling between the hEB pair by continuous propagation from one hEB to the other. FIG. 13C shows Vm traces (from the three boxes in a) demonstrate the synchrony of the action potentials, as the electrical wave propagates from right to left (red to blue to green trace) across the field of view.

FIG. 14 is a table showing the optimization of xeno- and serum-free day 2-4 media formulation. The optimal formulation for d2-d4 xeno- and serum-free differentiation was: (a) 5 mg mL⁻¹ HSA. (f) 280 μM ascorbic acid. (g) 1× lipids. (d) As with the xeno-containing d2-d4 media formulation, the addition of insulin inhibited cardiac differentiation.

### DETAILED DESCRIPTION OF THE INVENTION

The invention features a culture system, culture system components and culture methods that are useful for generating differentiated mesodermal cells.

The invention is based, at least in part, on the discovery of a highly efficient methodology for cardiac differentiation of human embryonic stem cells (hESC) and human induced pluripotent stem cells (hiPSC) that eliminates variability in differentiation capacity between cell lines. This system was systematically and rigorously optimized by modifying >45 experimental variables to develop a universal cardiac differentiation protocol that produces contracting human embryoid bodies (hEB) with a near total efficiency of 94.7±2.4% in an accelerated period of nine days in each of eight hESC/hiPSC lines tested. This cost-effective method employs forced aggregation hEB formation in a chemically defined medium along with staged exposure to physiological oxygen tension, titrated concentrations of mesodermal morphogens (BMP4, FGF2), polyvinyl alcohol, serum, and insulin. The contracting hEB derived using these methods displayed properties of functional cardiomyocytes including ultra-structural phenotypes, highly reproducible electrophysiological profiles and responsiveness to known cardioactive drugs. The efficiency and reproducibility of this method facilitates the application of hiPSC-derived cardiomyocytes to patient-specific cardiotoxicity drug testing, disease modeling, and cardiac regeneration. Advantageously, the culture system is free of animal products so the differentiated mesodermal cells are suitable for human cell therapy, as well as virtually any other research, clinical, therapeutic or prophylactic method where differentiated mesodermal cells are used. In particular embodiments, the invention provides comprising at least about 10% differentiated cardiac myocytes that is useful for the prevention, treatment or repair of a damaged cardiac tissue.

### Differentiated Mesodermal Cells

As reported herein, differentiated mesodermal cells (e.g., cardiac myocytes) are generated using the culture system and methods of the invention from any of a variety of human embryonic stem cells and human induced pluripotent stem cells. The invention provides methods for generating a differentiated mesodermal cell. In general, the method involves culturing a human stem cell as a monolayer on a proteinaceous cell culture matrix that supports cell adhesion (e.g., Matrigel™, Geltrex™ Reduced Growth Factor Basement Membrane Matrix or CELLstart humanized substrate for pluripotent cell culture); disaggregation of cells with 0.05% trypsin or TrypLE™; Accutase™, induction of cellular aggregated to promote human embryoid body formation, which is initiated by seeding actively growing cells in V-bottom or U-bottom 96- or 384-well plates; promotion of the aggregation of the cells is performed by seeding culturing the cells in media comprising poly(vinyl alcohol), bone morphogenic protein 4 (BMP4), and fibroblast growth factor 2 (FGF2); Optionally, cell aggregation is promoted by centrifuging the cells at 200 g to 1000 g; after the initial forty-eight hours which induce aggregation and mesodermal lineage differentiation, cells are transferred to conditions containing fetal bovine serum (FBS) or FBS-substitute that support mesodermal lineage specification; finally cells are transferring to U-bottomed or flat-bottomed tissue culture treated plates and maintaining the cells for six additional days under conditions that promote human embryoid body maturation.

In one embodiment, aggregation was promote using 100 μl per well of RPMI supplemented with 25 ng mL⁻¹ BMP, 5 ng mL⁻¹ FGF2 and 1 mg mL⁻¹ PVA (RPMI-PVA); mesoderm lineage specification was promoted by using 100 μl per well of RPMI supplemented with 20% FBS (RPMI-FBS). In another embodiment mesoderm lineage specification was promoted using 100 μl per well of the FBS-substitute media StemPro™-34 supplemented with ascorbic acid. On day 4 of differentiation the human embryoid bodies may be re-suspended in 100 μl of either RPMI-PVA or RPMI-FBS or StemPro™-34. In one embodiment differentiation is detected at day 7 when human embryoid bodies began contracting (beating). Identification of the percentage of beating hEBs indicates the efficiency of differentiation.

If desired, commercially available media is used in the methods of the invention, including mTeSR1, TeSR2 (Stem Cell Technologies), Nutristem (Stemgent), StemPro hESC SFM (Invitrogen) or media supplemented with Xeno-Free Knockout Serum Replacement and Xeno-Free Growth Factor Cocktail (Invitrogen).

More specifically, the invention provides method for generating a differentiated cardiac myocyte cell. The method generally involves culturing a human stem cell or human induced pluripotent stem cell in medium conditioned on irradiated mouse embryonic fibroblasts seeded at 6×10⁴ cells/cm² for 22-26 hour, where the conditioned media comprises at least about 4 ng/ml FGF2; culturing a human stem cell or human induced pluripotent stem cell of step a as a monolayer on a proteinaceous cell culture matrix that supports cell adhesion; plating cells from step b at about 5000 cells per well of a 96-well plate in culture media comprising 25 ng ml⁻¹ BMP4, 5 ng ml⁻¹ FGF2 and cell culture media comprising at least about 0.1% PVA and centrifuging the culture to force the cells to aggregate; maintaining the cells in culture for another forty-eight hours in media comprising media comprising FBS or a FBS-substitute, thereby promoting mesodermal lineage specification; and maintaining the cells for six additional days in media comprising 0.4% PVA or media comprising FBS or an FBS-substitute thereby generating a differentiated mesodermal cell. In one embodiment, the media used to promote differentiation is RPMI-PVA medium that comprises or consists essentially of RPMI with 4 mg mL⁻¹ PVA, 1× Lipid Concentrate, 400 mM 1-thioglycerol, and 10 ug/mL insulin. One of skill in the art will appreciate that the steps recited may be carried out either serially or in any combination. None of the steps are essential in the methods of the invention.

Human embryonic stem cells and human induced pluripotent stem cells are commercially available (e.g., from WiCell, which provides iPS(IMR-90)-1, iPS(IMR-90)-4 and iPS(Foreskin)-1). Human induced pluripotent stem cells can also be generated using methods known in the art from a variety of somatic cell types (Yu, J., K. Hu, et al. (2009). “Human induced pluripotent stem cells free of vector and transgene sequences.” Science 324(5928): 797-801.) Somatic cells particularly useful in the methods of the invention include but are not limited to fibroblasts, keratinocytes, foreskin fibroblasts, adipocytes, cord blood, mobilized peripheral blood, fetal liver blood, bone marrow aspirates. Other cells useful in the methods of the invention include embryonic stem cells, mesodermal stem cells, mesenchymal stem cells and all those known in the art that have been identified in mammalian organs or tissues.

The embryonic stem (ES) cell has unlimited self-renewal and pluripotent differentiation potential (Thomson, J. et al. 1995; Thomson, J. A. et al. 1998; Shamblott, M. et al. 1998; Williams, R. L. et al. 1988; Orkin, S. 1998; Reubinoff, B. E., et al. 2000). These cells are derived from the inner cell mass (ICM) of the pre-implantation blastocyst (Thomson, J. et al. 1995; Thomson, J. A. et al. 1998; Martin, G. R. 1981), or can be derived from the primordial germ cells from a post-implantation embryo (embryonal germ cells or EG cells). ES and/or EG cells have been derived from multiple species, including mouse, rat, rabbit, sheep, goat, pig and more recently from human and human and non-human primates (U.S. Pat. Nos. 5,843,780 and 6,200,806).

Embryonic stem cells are well known in the art. For example, U.S. Pat. Nos. 6,200,806 and 5,843,780 refer to primate, including human, embryonic stem cells. U.S. Patent Applications Nos. 20010024825 and 20030008392 describe human embryonic stem cells. U.S. Patent Application No. 20030073234 describes a clonal human embryonic stem cell line. U.S. Pat. No. 6,090,625 and U.S. Patent Application No. 20030166272 describe an undifferentiated cell that is stated to be pluripotent. U.S. Patent Application No. 20020081724 describes what are stated to be embryonic stem cell derived cell cultures.

Stem cells of the present invention also include mesodermal stem cells. Mesenchymal stem cells, or “MSCs” are well known in the art. MSCs, originally derived from the embryonal mesoderm and isolated from adult bone marrow, can differentiate to form muscle, bone, cartilage, fat, marrow stroma, and tendon. During embryogenesis, the mesoderm develops into limb-bud mesoderm, tissue that generates bone, cartilage, fat, skeletal muscle and endothelium. Mesoderm also differentiates to visceral mesoderm, which can give rise to cardiac muscle, smooth muscle, or blood islands consisting of endothelium and hematopoietic progenitor cells. Primitive mesodermal or MSCs, therefore, could provide a source for a number of cell and tissue types. A number of MSCs have been isolated. (See, for example, Caplan, A., et al., U.S. Pat. No. 5,486,359; Young, H., et al., U.S. Pat. No. 5,827,735; Caplan, A., et al., U.S. Pat. No. 5,811,094; Bruder, S., et al., U.S. Pat. No. 5,736,396; Caplan, A., et al., U.S. Pat. No. 5,837,539; Masinovsky, B., U.S. Pat. No. 5,837,670; Pittenger, M., U.S. Pat. No. 5,827,740; Jaiswal, N., et al., (1997). J. Cell Biochem. 64(2):295-312; Cassiede P., et al., (1996). J Bone Miner Res. 9:1264-73; Johnstone, B., et al., (1998) Exp Cell Res. 1:265-72; Yoo, et al., (1998) J Bon Joint Surg Am. 12:1745-57; Gronthos, S., et al., (1994). Blood 84:4164-73); Pittenger, et al., (1999). Science 284:143-147.

Biological samples may comprise mixed populations of cells, which can be purified to a degree sufficient to produce a desired effect. Those skilled in the art can readily determine the percentage of differentiated cells (e.g., differentiated mesodermal cells, cardiac myocytes) or their progenitors in a population using various well-known methods, such as fluorescence activated cell sorting (FACS). Purity of differentiated cells can be determined according to the genetic marker profile within a population, using immunostaining, using cell morphology, by determining the number of cells that “beat.”, or by detecting eletrophysiological profile. In several embodiments, it will be desirable to purify the cells before, during, or after the differentiation protocol. Differentiated mesodermal cells of the invention preferably comprise a population of cells that have about 50-55%, 55-60%, 60-65% and 65-70% purity (e.g., undifferentiated cells have been removed or are otherwise absent from the population). More preferably the purity is about 70-75%, 75-80%, 80-85%; and most preferably the purity is about 85-90%, 90-95%, and 95-100%.

### Pharmaceutical Compositions Comprising Differentiated Mesodermal Cells

A differentiated mesodermal cell (e.g., cardiac myocyte) of the invention may be combined with pharmaceutical excipients known in the art to enhance preservation and maintenance of the cells prior to administration. In some embodiments, cell compositions of the invention can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Sterile injectable solutions can be prepared by incorporating the cells utilized in practicing the present invention in the required amount of the appropriate solvent with various amounts of the other ingredients, as desired. Such compositions may be in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. The compositions can be isotonic, i.e., they can have the same osmotic pressure as blood and lacrimal fluid. The desired isotonicity of the compositions of this invention may be accomplished using sodium chloride, or other pharmaceutically acceptable agents such as dextrose, boric acid, sodium tartrate, propylene glycol or other inorganic or organic solutes. Sodium chloride is preferred particularly for buffers containing sodium ions.

A method to potentially increase cell survival when introducing the cells into a subject in need thereof is to incorporate stem cells of interest into a biopolymer or synthetic polymer. Depending on the subject's condition, the site of injection might prove inhospitable for cell seeding and growth because of scarring or other impediments. Examples of biopolymer include, but are not limited to, cells mixed with fibronectin, fibrin, fibrinogen, thrombin, collagen, and proteoglycans. This could be constructed with or without included expansion or differentiation factors. Additionally, these could be in suspension, but residence time at sites subjected to flow would be nominal. Another alternative is a three-dimensional gel with cells entrapped within the interstices of the cell biopolymer admixture. Again, expansion or differentiation factors could be included with the cells. These could be deployed by injection via various routes described herein.

Those skilled in the art will recognize that the components of the compositions should be selected to be chemically inert and will not affect the viability or efficacy of the stem cells or their progenitors as described in the present invention. This will present no problem to those skilled in chemical and pharmaceutical principles, or problems can be readily avoided by reference to standard texts or by simple experiments (not involving undue experimentation), from this disclosure and the documents cited herein.

One consideration concerning the therapeutic use of stem cells is the quantity of cells necessary to achieve an optimal effect. Different scenarios may require optimization of the amount of cells injected into a tissue of interest. Thus, the quantity of cells to be administered will vary for the subject being treated. The precise determination of what would be considered an effective dose may be based on factors individual to each patient, including their size, age, sex, weight, and condition of the particular patient. As few as 100-1000 or as many as 1,000,000-1,000,000,000 cells can be administered for certain desired applications among selected patients. Therefore, dosages can be readily ascertained by those skilled in the art from this disclosure and the knowledge in the art.

The skilled artisan can readily determine the amount of cells and optional additives, vehicles, and/or carrier in compositions and to be administered in methods of the invention. Of course, for any composition to be administered to an animal or human, and for any particular method of administration, it is preferred to determine therefore: toxicity, such as by determining the lethal dose (LD) and LD50 in a suitable animal model e.g., rodent such as mouse; and, the dosage of the composition(s), concentration of components therein and timing of administering the composition(s), which elicit a suitable response. Such determinations do not require undue experimentation from the knowledge of the skilled artisan, this disclosure and the documents cited herein. And, the time for sequential administrations can be ascertained without undue experimentation.

### Tissue Repair

The culture system of the invention provides for the rapid production of cardiomyocytes from pluripotent cells. Moreover, cardiomyocytes comprise at least about 75%, 80%, 85%, 90%, 95% or even 100% of the differentiated cells in vitro. In one embodiment, the culture system of the invention preferably provides at least about 90% differentiated mesodermal cells, such as cardiomyocytes. Because such cells are generated without animal products (e.g., bovine serum albumin), formulations comprising such cells provide GMP-grade cardiomyocytes for clinical use. Accordingly, the invention features compositions and methods for repairing damaged tissues using differentiated mesodermal cells, such as cardiac myocytes. Tissues amenable to treatment using the cells of the invention, including cardiac myocytes, include cardiac tissue damaged by myocardial infarction or heart failure or by cardiomyopathies such as congenital heart disease, nutritional diseases, ischemic (or non-ischaemic) cardiomyopathy, hypertensive cardiomyopathy, valvular cardiomyopathy, inflammatory cardiomyopathy, cardiomyopathy secondary to a systemic metabolic disease, alcoholic cardiomyopathy, diabetic cardiomyopathy, restrictive cardiomyopathy. Therapeutic compositions comprising the cells are administered to a damaged or diseased tissue.

### Administration of Differentiated Mesodermal Cells

Differentiated mesodermal cells (e.g. cardiac myocytes) of the invention are administered according to methods known in the art. Such compositions may be administered by any conventional route, including injection or by gradual infusion over time. The administration may, depending on the composition being administered, for example, be, intravenous, intraperitoneal, intramuscular, intracavity, subcutaneous, or transdermal. The cells are administered in “effective amounts”, or the amounts that either alone or together with further doses produces the desired therapeutic response.

Administered cells of the invention can be autologous (“self”) or non-autologous (“non-self,” e.g., allogeneic, syngeneic or xenogeneic). Generally, administration of the cells can occur within a short period of time following differentiation in culture (e.g. 1, 2, 5, 10, 24 or 48 hours after completion of the culture protocol) or the mature mesodermal cells can be maintained in culture for days, weeks (e.g., 1, 2, 3, 4, 5, 6) or even months (e.g., 1, 2, 3, 4, 5, 6, 9, 12, 18, 24) and according to the requirements of each desired treatment regimen.

Compositions comprising a differentiated mesodermal cell (e.g., cardiac myocyte) are provided systemically or directly to a site of injury. Modes of administration include intramuscular, intra-cardiac, oral, rectal, topical, intravaginal, intracisternal, intratracheal, nasal, transdermal, within/on implants, e.g., fibers such as collagen, osmotic pumps, or parenteral routes. The term “parenteral” includes subcutaneous, intravenous, intramuscular, intraperitoneal, intragonadal or infusion.

In one approach, cells derived from cultures of the invention are implanted into a host. In particular embodiments, at least 100,000, 250,000, or 500,000 cells is injected. In other embodiments, 750,000, or 1,000,000 cells is injected. In other embodiments, at least about 1×10⁵ cells will be administered, 1×10⁶, 1×10⁷, or even as many as 1×10⁸ to 1×10¹°, or more are administered.

Selected cells of the invention comprise a purified population of differentiated mesodermal cells (e.g., cardiac myocytes). Those skilled in the art can readily determine the percentage of cells in a population using various well-known methods, such as fluorescence activated cell sorting (FACS). Preferable ranges of purity in populations comprising selected cells are about 50 to about 55%, about 55 to about 60%, and about 65 to about 70%. More preferably the purity is at least about 70%, 75%, or 80% pure, more preferably at least about 85%, 90%, or 95% pure. In some embodiments, the population is at least about 95% to about 100% differentiated mesodermal cells (e.g., cardiac myocytes). Dosages can be readily adjusted by those skilled in the art (e.g., a decrease in purity may require an increase in dosage). The cells can be introduced by injection, catheter, or the like. Compositions of the invention include pharmaceutical compositions comprising a differentiated mesodermal cell (e.g., cardiac myocyte) and a pharmaceutically acceptable carrier.

Differentiated mesodermal cells (e.g., cardiac myocytes) an be administered via localized injection, including catheter administration, systemic injection, localized injection, intravenous injection, or parenteral administration. When administering a therapeutic composition of the present invention, it will generally be formulated in a unit dosage injectable form (solution, suspension, emulsion).

If desired, a differentiated mesodermal cell (e.g., cardiac myocyte) is incorporated into a polymer scaffold to promote tissue repair, cell survival, proliferation in a tissue in need thereof. Polymer scaffolds can comprise, for example, a porous, non-woven array of fibers. The polymer scaffold can be shaped to maximize surface area, to allow adequate diffusion of nutrients and growth factors to a cell of the invention. Polymer scaffolds can comprise a fibrillar structure. The fibers can be round, scalloped, flattened, star-shaped, solitary or entwined with other fibers. Branching fibers can be used, increasing surface area proportionately to volume.

Unless otherwise specified, the term “polymer” includes polymers and monomers that can be polymerized or adhered to form an integral unit. The polymer can be non-biodegradable or biodegradable, typically via hydrolysis or enzymatic cleavage. The term “biodegradable” refers to materials that are bioresorbable and/or degrade and/or break down by mechanical degradation upon interaction with a physiological environment into components that are metabolizable or excretable, over a period of time from minutes to three years, preferably less than one year, while maintaining the requisite structural integrity. As used in reference to polymers, the term “degrade” refers to cleavage of the polymer chain, such that the molecular weight stays approximately constant at the oligomer level and particles of polymer remain following degradation.

Materials suitable for polymer scaffold fabrication include polylactic acid (PLA), poly-L-lactic acid (PLLA), poly-D-lactic acid (PDLA), polyglycolide, polyglycolic acid (PGA), polylactide-co-glycolide (PLGA), polydioxanone, polygluconate, polylactic acid-polyethylene oxide copolymers, modified cellulose, collagen, polyhydroxybutyrate, polyhydroxpriopionic acid, polyphosphoester, poly(alpha-hydroxy acid), polycaprolactone, polycarbonates, polyamides, polyanhydrides, polyamino acids, polyorthoesters, polyacetals, polycyanoacrylates, degradable urethanes, aliphatic polyester polyacrylates, polymethacrylate, acyl substituted cellulose acetates, non-degradable polyurethanes, polystyrenes, polyvinyl chloride, polyvinyl flouride, polyvinyl imidazole, chlorosulphonated polyolifins, polyethylene oxide, polyvinyl alcohol, teflon RTM, nylon silicon, and shape memory materials, such as poly(styrene-block-butadiene), polynorbornene, hydrogels, metallic alloys, and oligo(ε-caprolactone)diol as switching segment/oligo(p-dioxyanone)diol as physical crosslink. Other suitable polymers can be obtained by reference to The Polymer Handbook, 3rd edition (Wiley, N.Y., 1989).

### Methods for Evaluating Therapeutic Efficacy

In one approach, the efficacy of the treatment is evaluated by measuring, for example, the biological function of the treated organ. For example, the efficacy of treatment is evaluated by monitoring cardiac function before and after the administration of a differentiated cardiac myocyte. In one embodiment, cardiac function is assayed by electrocardiogram, functional magnetic resonance imaging (fMRI) or positronic eletro PET or by measuring ejection fraction by electrocardiogram. In other embodiments, the biological function of bladder, bone, brain, breast, cartilage, esophagus, fallopian tube, heart, pancreas, intestines, gallbladder, kidney, liver, lung, nervous tissue, ovaries, prostate, skeletal muscle, skin, spinal cord, spleen, stomach, testes, thymus, thyroid, trachea, ureter, urethra, urogenital tract, or uterus is assayed. Methods for evaluating the biological function of tissues and/or organs are standard in the art and are described, for example, in the Textbook of Medical Physiology, Tenth edition, (Guyton et al., W.B. Saunders Co., 2000). Preferably, a method of the present invention, increases the biological function of a tissue or organ by at least 5%, 10%, 20%, 40%, 50%, 60%, 70%, 80%, 90%, 100%, 150%, 200%, or even by as much as 300%, 400%, or 500%.

In another approach, the therapeutic efficacy of the methods of the invention is assayed by measuring an increase in cell number in the treated or transplanted tissue or organ as compared to a corresponding control tissue or organ (e.g., a tissue or organ that did not receive treatment). Preferably, cell number in a tissue or organ is increased by at least 5%, 10%, 20%, 40%, 60%, 80%, 100%, 150%, or 200% relative to a corresponding tissue or organ. Methods for assaying cell proliferation are known to the skilled artisan and are described, for example, in Bonifacino et al., (Current Protocols in Cell Biology Loose-leaf, John Wiley and Sons, Inc., San Francisco, Calif.).

In another approach, efficacy is measured by detecting an increase in the number of viable cells present in a tissue or organ relative to the number present in an untreated control tissue or organ, or the number present prior to treatment. Assays for measuring cell viability are known in the art, and are described, for example, by Crouch et al. (J. Immunol. Meth. 160, 81-8); Kangas et al. (Med. Biol. 62, 338-43, 1984); Lundin et al., (Meth. Enzymol. 133, 27-42, 1986); Petty et al. (Comparison of J. Biolum. Chemilum. 10, 29-34, 1995); and Cree et al. (AntiCancer Drugs 6: 398-404, 1995). Cell viability can be assayed using a variety of methods, including MTT (3-(4,5-dimethylthiazolyl)-2,5-diphenyltetrazolium bromide) (Barltrop, Bioorg. & Med. Chem. Lett. 1: 611, 1991; Cory et al., Cancer Comm. 3, 207-12, 1991; Paull J. Heterocyclic Chem. 25, 911, 1988). Assays for cell viability are also available commercially. These assays include but are not limited to CELLTITER-GLO® Luminescent Cell Viability Assay (Promega), which uses luciferase technology to detect ATP and quantify the health or number of cells in culture, and the CellTiter-Glo® Luminescent Cell Viability Assay, which is a lactate dehyrodgenase (LDH) cytotoxicity assay (Promega).

Cardiac Drug Screening [***Drs. Zambidis and Burridge: Please Confirm or Elaborate.***]

The differentiated cardiac myocytes of the invention are particularly useful in drug screening applications. Cells of the invention provide a virtually unlimited supply of cardiac cells that can be used, for example, to test the effects of various agents (e.g., compounds, peptides, polynucleotides) on cardiac biological activity. In one embodiment, cells of the invention are cultured and an agent of interest is added to the culture media. The effect of the agent on cardiac biological activity is then assessed using any method known in the art. In one embodiment, the cardiac toxicity of the agent is assessed by assaying the culture for alterations in cardiac cell viability. Agents that increase cardiac cell death, reduce cardiac cell viability, or otherwise reduce cardiac cell biological activity are identified as having undesirable cardiac toxicity. In another embodiment, the effect of the agent is assessed by assaying a biological activity of a cardiac cell, wherein an agent that increases the biological activity is identified as useful for treating a cardiac condition. If desired, cardiac cells of the invention are engineered to express a protein of interest that increases or decreases a cardiac function. Such cells may also be used in drug screening for the identification of agents as cardiotoxic or as having a desirable effect on cardiac function.

### Kits

The culture system of the invention can be supplied in the form of a kit comprising one or more tissue culture reagents (e.g., culture media, culture flasks, plates) and directions for using the kit in any culture method delineated herein. In one embodiment, the directions provide for the generation of a differentiated mesodermal cell (e.g., cardiac myocyte). In another embodiment, the kit comprises a differentiated mesodermal cell (e.g., cardiac myocyte) and directions for use of the cell in a treatment method delineated herein. The kits can include instructions for the treatment regime, reagents, equipment (test tubes, reaction vessels, needles, syringes, etc.) and standards for calibrating or conducting the treatment. The instructions provided in a kit according to the invention may be directed to suitable operational parameters in the form of a label or a separate insert. Optionally, the kit may further comprise a standard or control information so that the test sample can be compared with the control information standard to determine if whether a consistent result is achieved.

In general, a culture system includes one or more containers of media, including cell culture media that promotes mesodermal lineage specification containing FBS or a FBS-substitute, about 25 ng ml⁻¹ BMP4 and about 5 ng ml⁻¹ FGF2; cell culture media that promotes cell aggregation containing about 25 ng ml⁻¹ BMP4, about 5 ng ml⁻¹ FGF2 and about 0.4% PVA; cell culture media for maintaining human embryonic stem cells or human induced pluripotent cells in culture, where the consists of a defined medium containing growth factors such FGF2 and/or TGFB and/or human or bovine serum albumin designed expressly for the maintenance of pluripotent stem cells or a medium that is conditioned on irradiated mouse embryonic fibroblasts seeded at about 6×10⁴ cells/cm² for about 22-26 hour or longer, and where the conditioned media contains at least about 4 ng/ml FGF2. If desired, the culture system further includes culture flasks or culture plates, including any of those delineated herein.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1

### A Systematic Strategy for Sequentially Optimizing Cardiac Differentiation

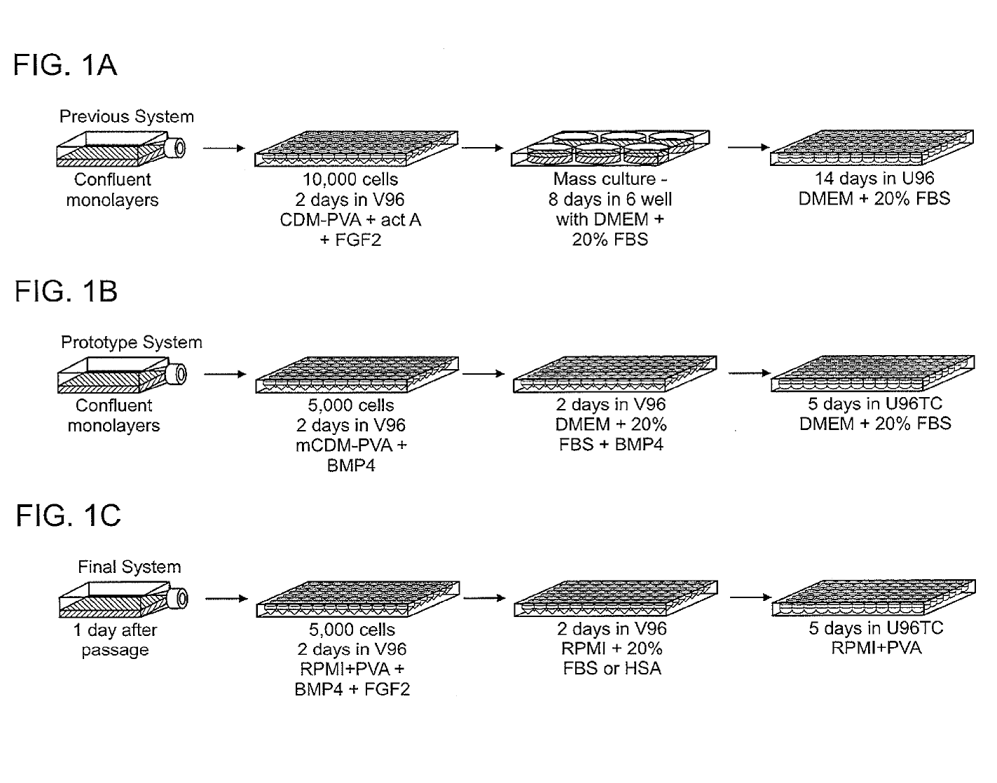

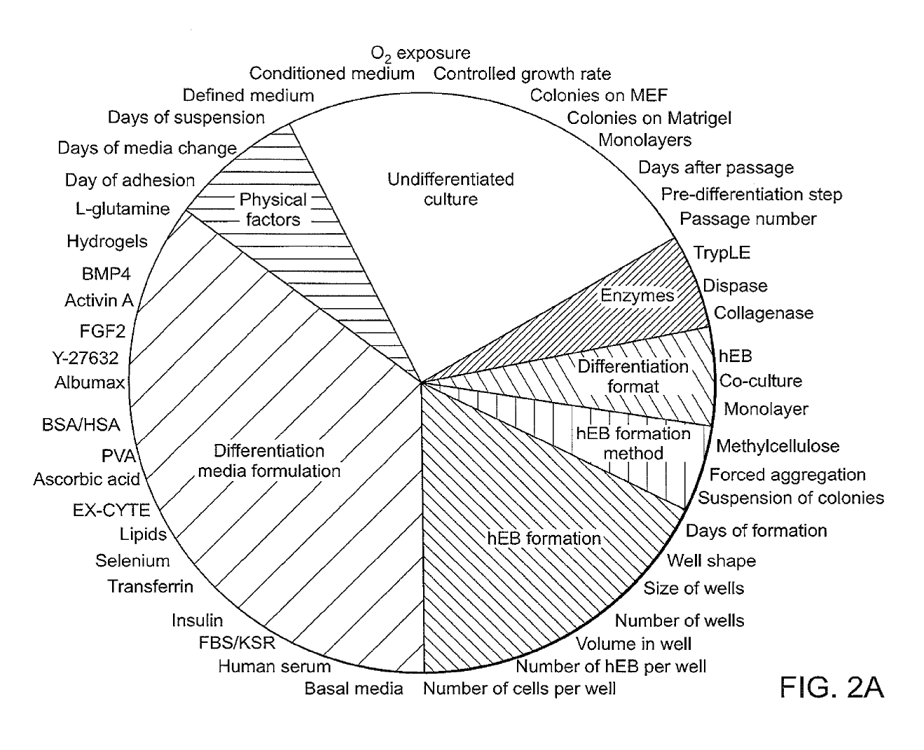

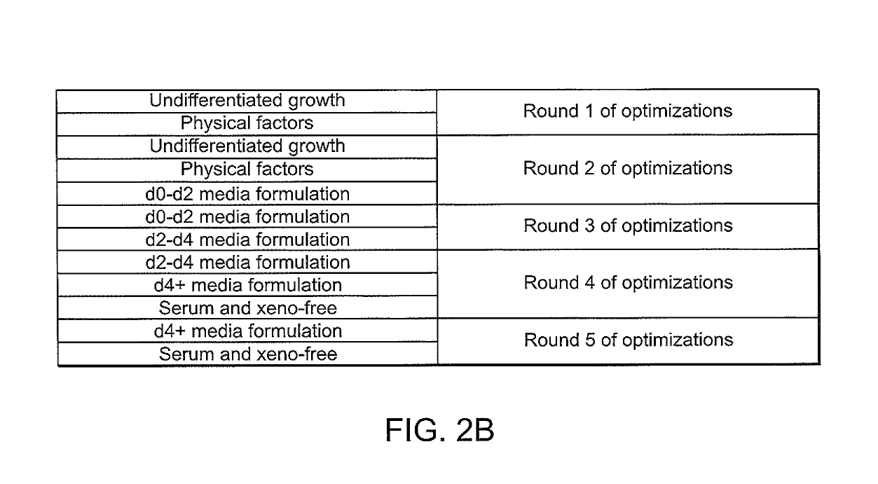

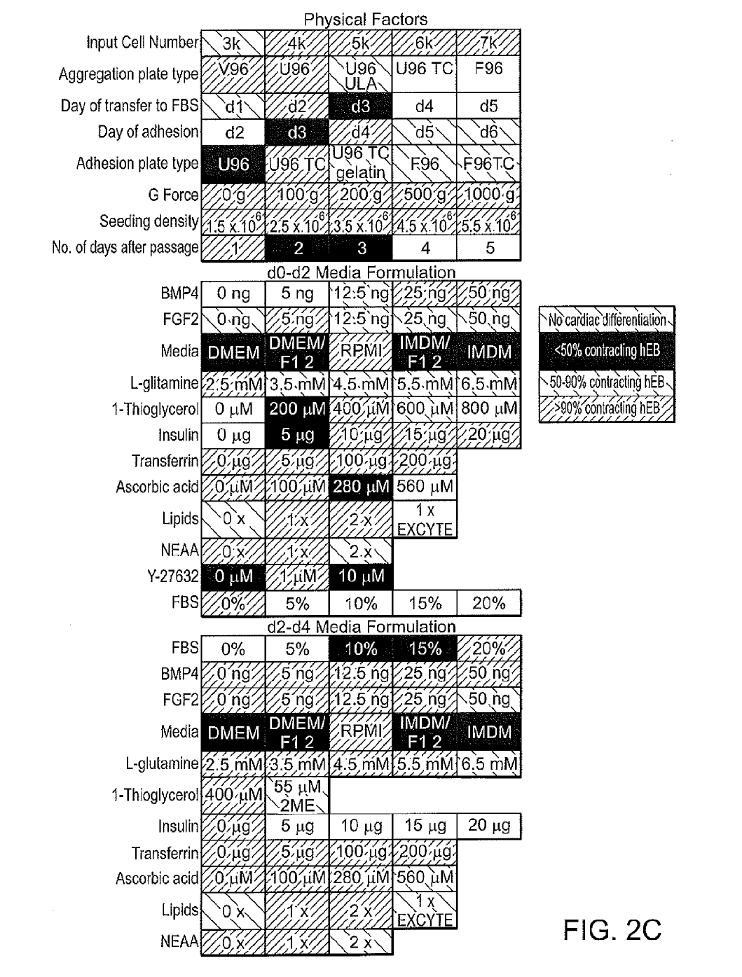

To improve the efficiency, reproducibility, and to reduce the interline variability of cardiac differentiation, cardiac differentiation strategies were analyzed (FIG. 1A-D), and experimental variables were identified (FIG. 2A). A strategy for systematically optimizing the cardiac differentiation of hPSC was then initiated (FIG. 2B). A forced aggregation cardiac differentiation system was employed to form uniform homogeneous hEB from known numbers of cells (Burridge et al., Stem Cells 25, 929-938 (2007)) (FIG. 1A). H9 (WA09) hESC, a cell line that has proven refractory to efficient cardiac lineage differentiation, was used for initial system development.

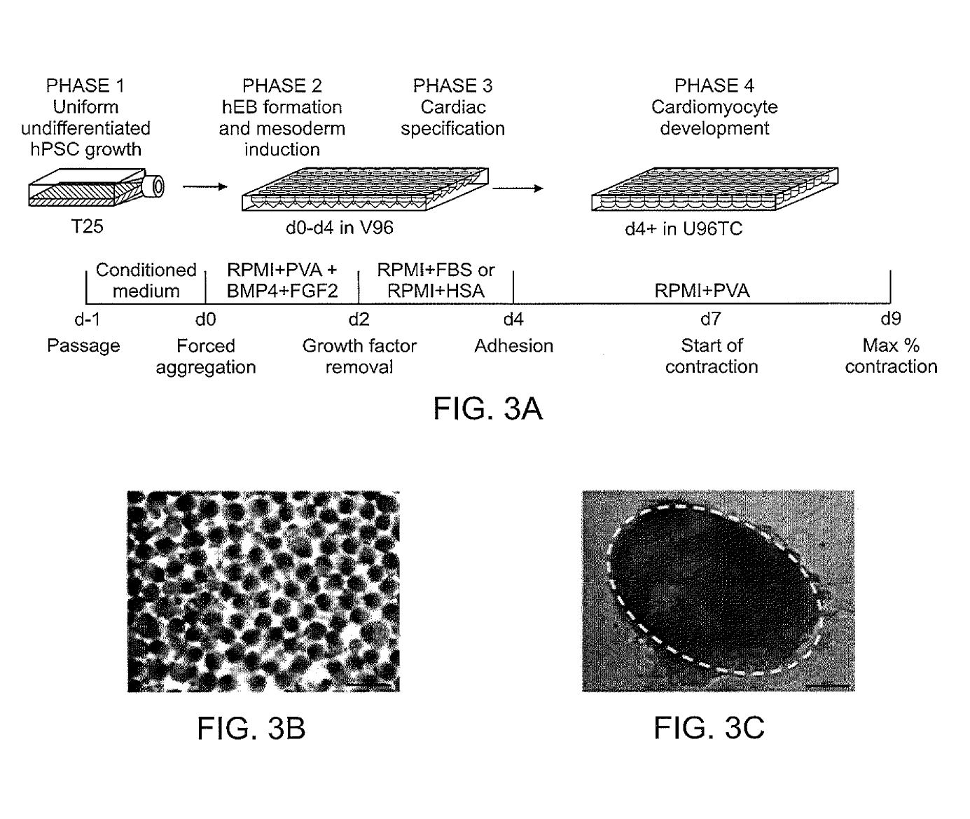

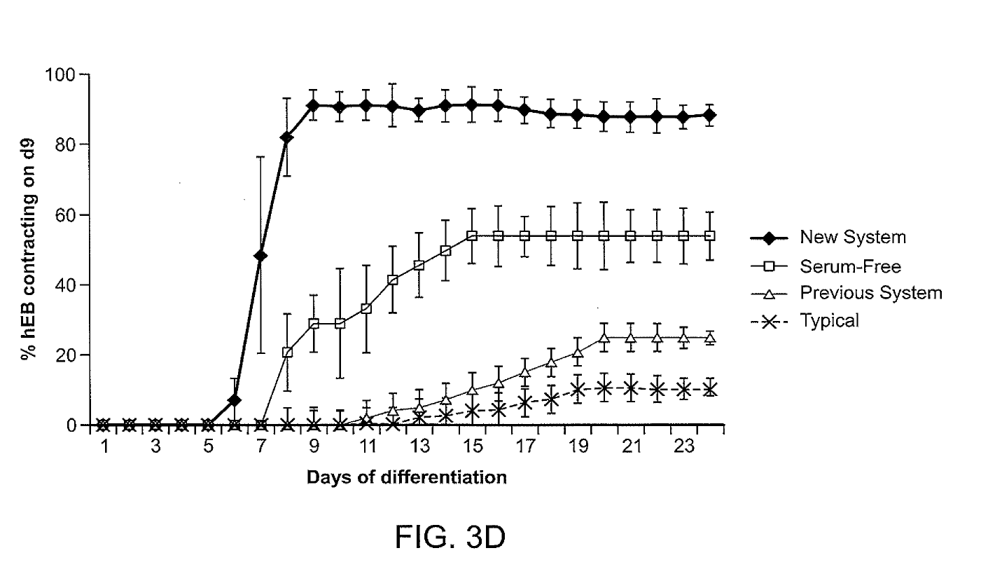

The cardiac differentiation protocol was divided into four distinct phases for optimization (FIG. 3A): phase 1: uniform undifferentiated growth; phase 2: hEB formation/mesoderm induction; phase 3: hEB cardiac specification; phase 4: contracting cardiomyocyte development. A simple beating hEB assay was used to sequentially assess improvements in cardiac differentiation in each of these four phases by counting the number of hEB contracting after nine days of differentiation, a time point that was identified as the day of maximum percentage contraction in the enhanced ‘prototype’ version of the protocol (FIG. 1B). After an initial round of optimizations had been completed, optimizations was repeated a second time to account for the new media formulations and then a third time to optimize the final media formulation. In some cases fourth and fifth repeats were performed to improve the statistical power of the results. For each variable tested, the bell-curve distribution of dose-response was determined. Results shown are averages of the 2nd-5th replicates (FIG. 2B).

The final fully-developed system (FIG. 3A and FIG. 1C) reproducibly formed homogeneous H9 hEB (FIG. 3B) which began contracting at a significantly (p<3×10-10) improved efficiency of 91.2±1.9% in an accelerated time period of only 9 days of differentiation compared to an average efficiency of 10.4±6.8% using traditional methods (FIG. 3D). Additionally, in contrast to prior methodologies that produce only rare, focused areas of contracting cells at the periphery of the hEB, the optimized differentiation method produced robust and forceful contractions within the entire hEB (FIG. 3C). Herein below is described the approach for the systematic optimization of each of the four phases of this highly efficient cardiac differentiation protocol.

Phase 1: Defined Single-Cell Culture Promotes Uniform Growth of hPSC Lines

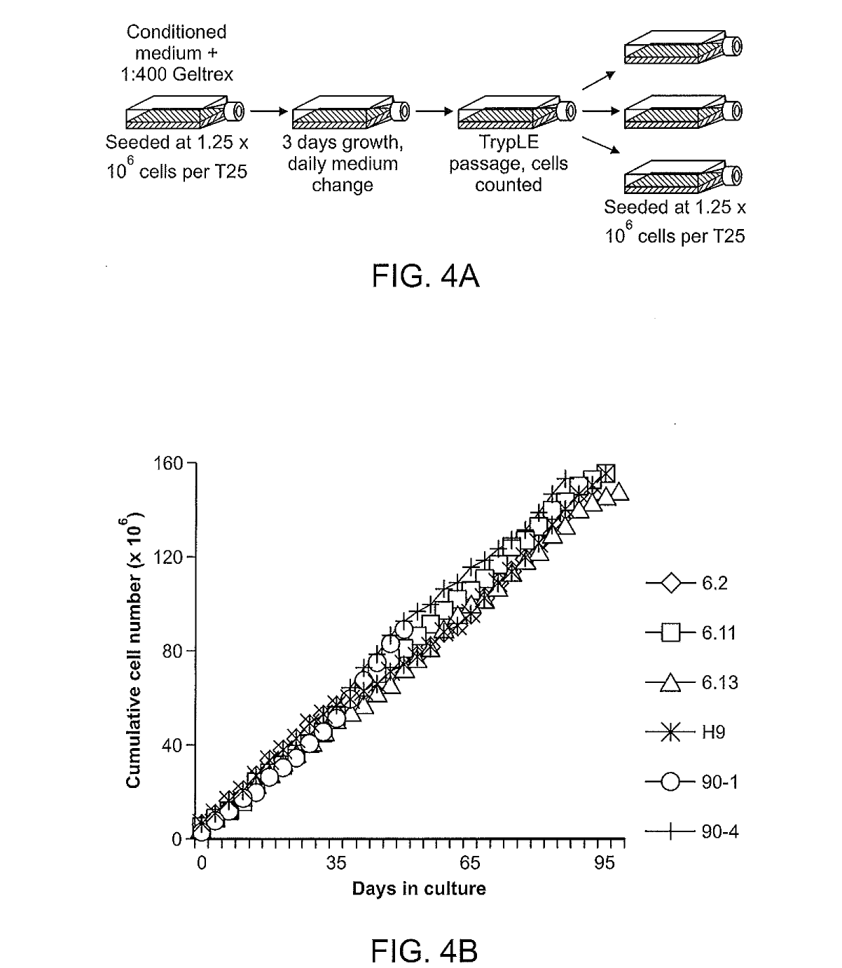

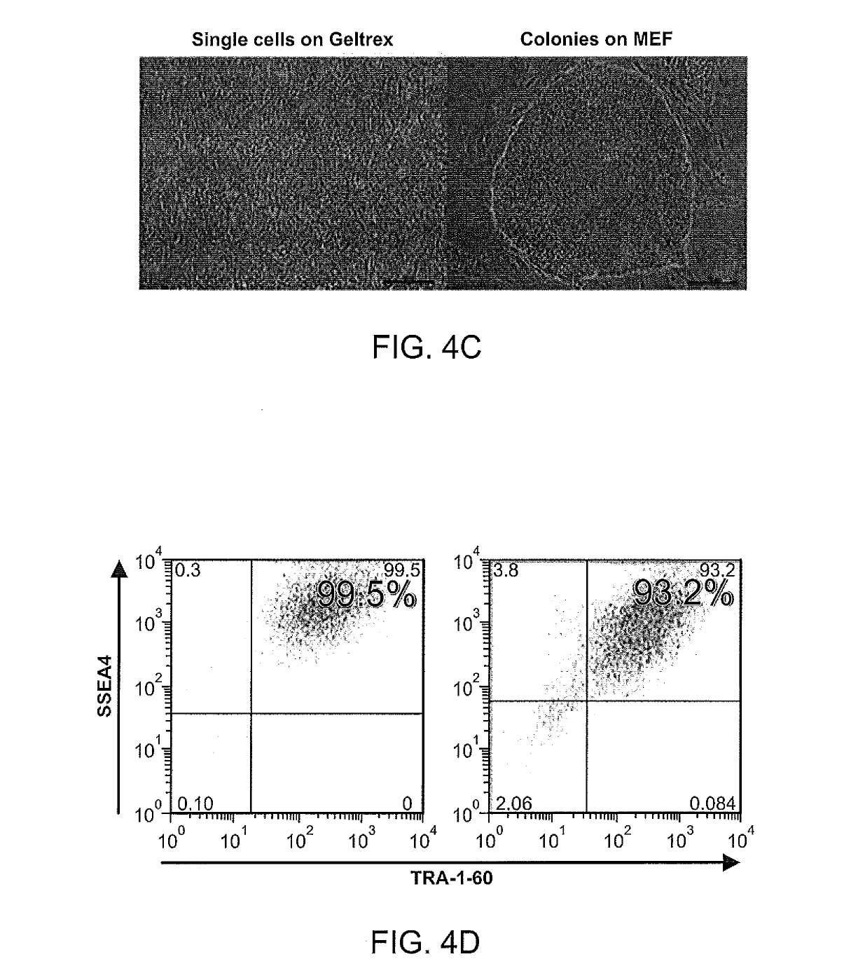

To determine whether promoting uniform growth of undifferentiated hPSC lines is important for subsequent reproducible differentiation, and for the derivation of a universal cardiac differentiation protocol, hPSC cells lines were adapted to a feeder-free monolayer growth technique. In this system, cells were enzymatically-passaged to single cells on a rigid timescale of every three days, counted using an automated cell counter and plated at fixed cell densities. (FIG. 4A). This approach resulted in controlled, reproducible growth of multiple hPSC lines including hESC lines H9, ES03, and SI-233 as well as viral fibroblast-derived hiPSC lines iPS(IMR90)-1 and iPS(IMR90)-4), and non-viral CD34+ cord blood-derived hiPSC lines 6.2, 6.11 and 6.13. This technique allowed stable growth of at least eight hESC/hiPSC lines for over 40 passages (FIG. 4B). hPSC lines cultured in this manner maintained high expression levels (>99%) of the pluripotency markers SSEA4 and TRA-1-60 (FIG. 4C).

Phase 2 (d0-d2): A Chemically Defined Medium Accelerates Mesoderm Induction

To improve the second phase of this protocol, hEB formation was optimized via forced aggregation (Burridge et al., Stem Cells 25, 929-938 (2007)) by systematically testing hESC and mESC differentiation and culture media formulations. Using 10,000 cells per hEB, one media formulation (Wiles and Johansson, Exp Cell Res 247, 241-248 (1999)) formed the most reproducibly homogeneous hEB via forced aggregation (FIG. 4A). The individual components of this media formulation were tested in an effort to further enhance cardiac differentiation. The final media formulation that gave the most efficient cardiac differentiation is described as RPMI+PVA (Table 1).

TABLE 1
Media Formulations

Phase 2 (d 0-d 2) medium
RPMI 1640
400 μM 1-Thioglycerol
4 mg mL⁻¹ PVA
10 μg mL⁻¹ hr-Insulin
25 ng mL⁻¹ BMP4
5 ng mL⁻¹ FGF2
1 × lipids
1 μM Y27632
Phase 3 (d 2-d 4) medium
RPMI 1640
400 μm 1-Thioglycerol
20% FBS or hSerum
Phase 3 (d 2-d 4) serum-free medium
RPMI 1640
400 uM 1-Thioglycerol
5 mg mL⁻¹ hSA
1 × lipids
280 μM L-ascorbic acid
Phase 4 (d 4+) medium
RPMI 1640
400 μm 1-Thioglycerol
4 mg mL⁻¹ PVA
10 μg mL⁻¹ hr-Insulin
1 × lipids

Table 1 shows the final optimized media formulations for each of the three steps of cardiac differentiation. Two different media formulations were used during the forced aggregation cardiac differentiation procedure that produces >90% of hEB contracting by d9 of differentiation in 8 hESC/hiPSC lines tested. Also included is a serum free version of the d2-d4 media which produces ˜60% of hEB contracting by d15 of differentiation.

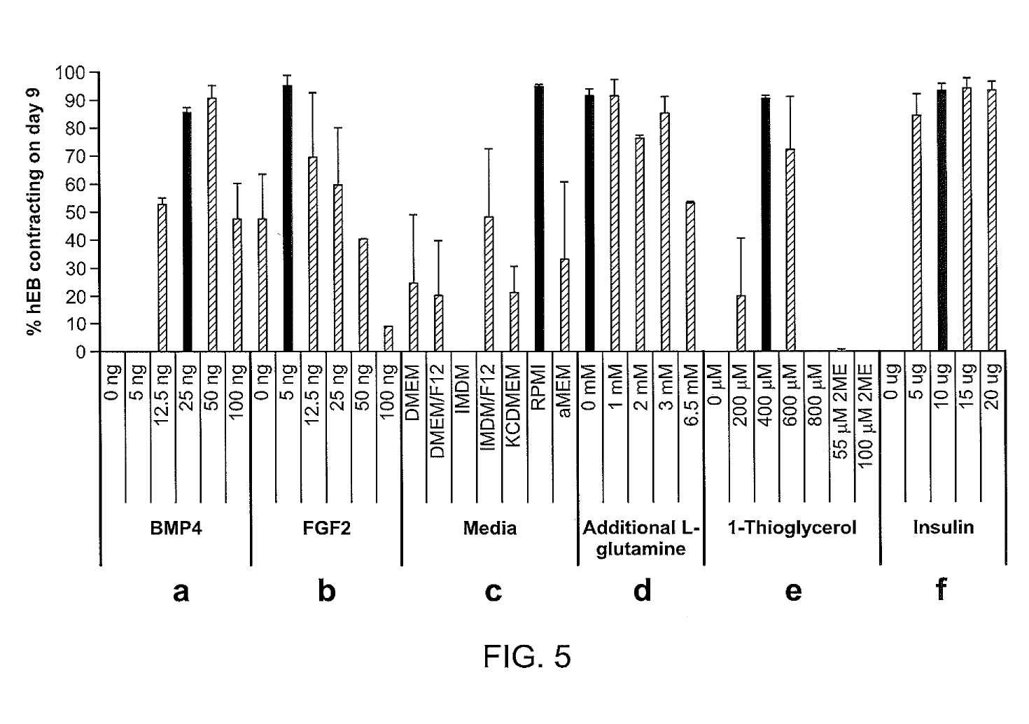

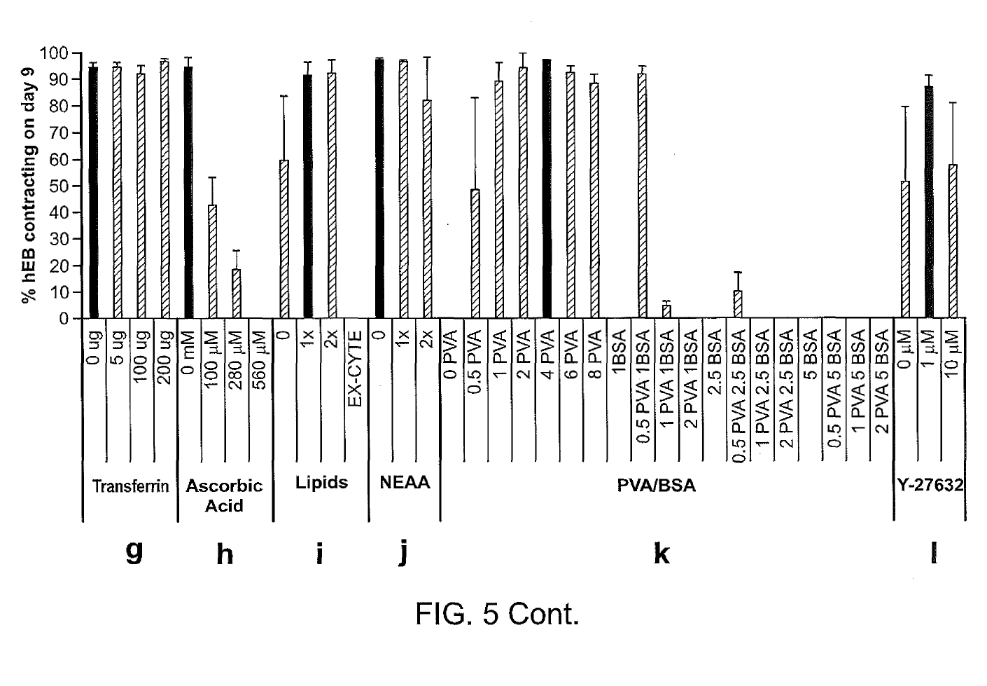

Three of the components of this media (RPMI, PVA, and insulin) were sufficient for forced aggregation hEB formation, but supplementation of this minimal formula with lipids and 1-thioglycerol resulted in high efficiency cardiac differentiation (FIGS. 5E, 5I). The inclusion of the small molecule Y-27632 (ROCK inhibitor) further improved reproducibility and cardiac differentiation. This was used at a limited concentration of 1 μM (FIG. 5I).

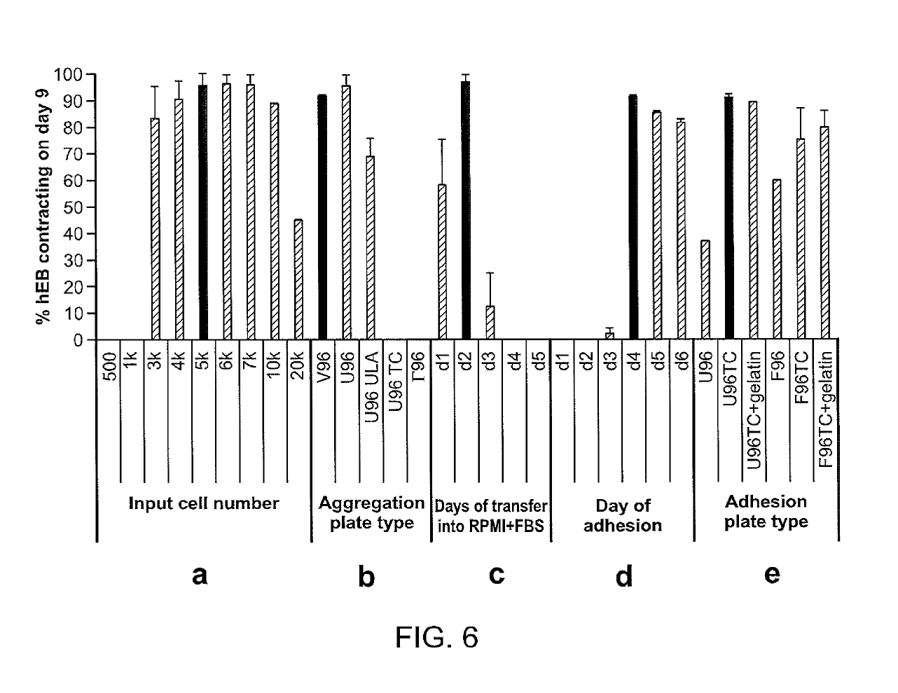

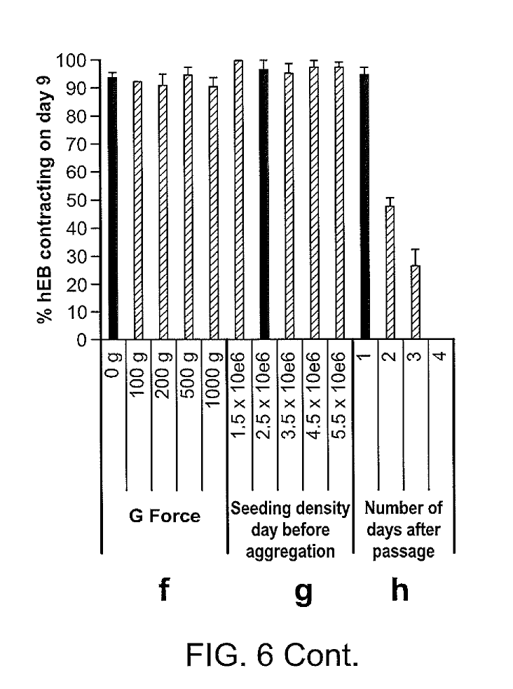

The effects of cell handling prior to and during forced aggregation was assessed. Cardiac differentiation of hEB made from monolayers of pluripotent cells passaged one day prior to forced aggregation was more efficient (93.75±3.3% contracting hEB) than those passaged 2 days (46.9±4.1%), or 3 days (26.0±8.0%) earlier (FIG. 6H). The number of cells seeded for hEB formation (ranging from 3,000-10,000 single hESC) had minimal effect on subsequent differentiation efficiency (FIG. 6A). The application of centrifugal force (g-force) to the 96-well plates during forced aggregation did not affect hEB formation in the presence of Y-27632 (FIG. 6F). Finally, the combination of the growth factors BMP4 and FGF2 resulted in optimally efficient cardiac differentiation (FIGS. 5A, 5B). Other growth factors including NODAL, activin A, TDGF1, BMP2, BMP6, TGFB, IGF1, IGF2 and WNT3A were each individually titrated between 1-100 ng mL-1 and none were found to have the same potency as BMP4.

Phase 3 (d2-d4): Efficient Cardiac Specification is Inhibited by Insulin and Potentiated by Serum

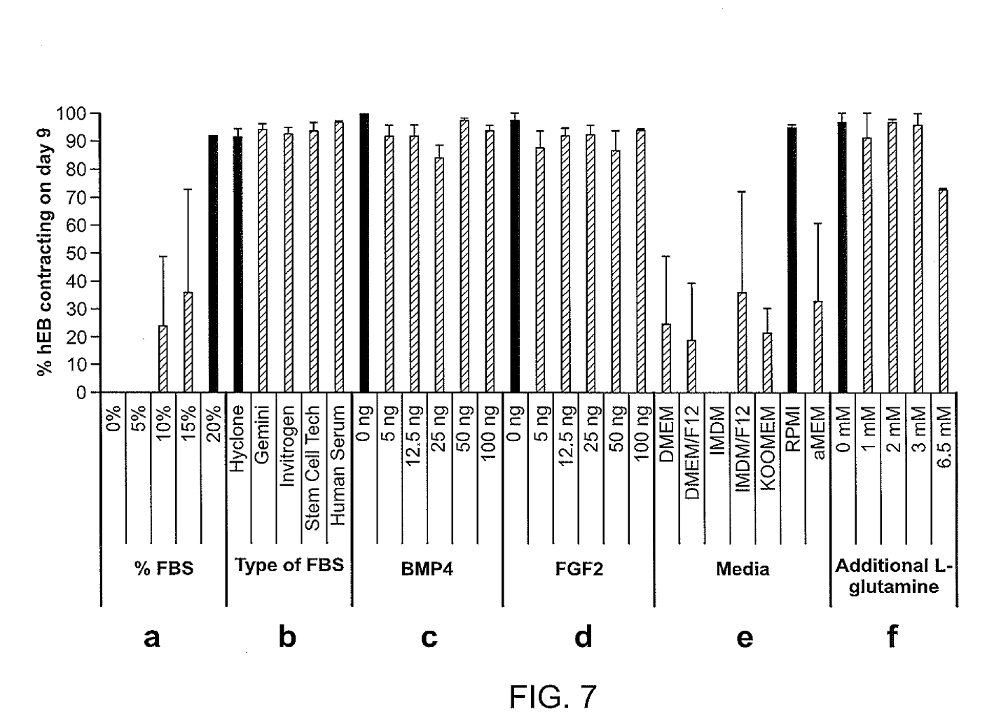

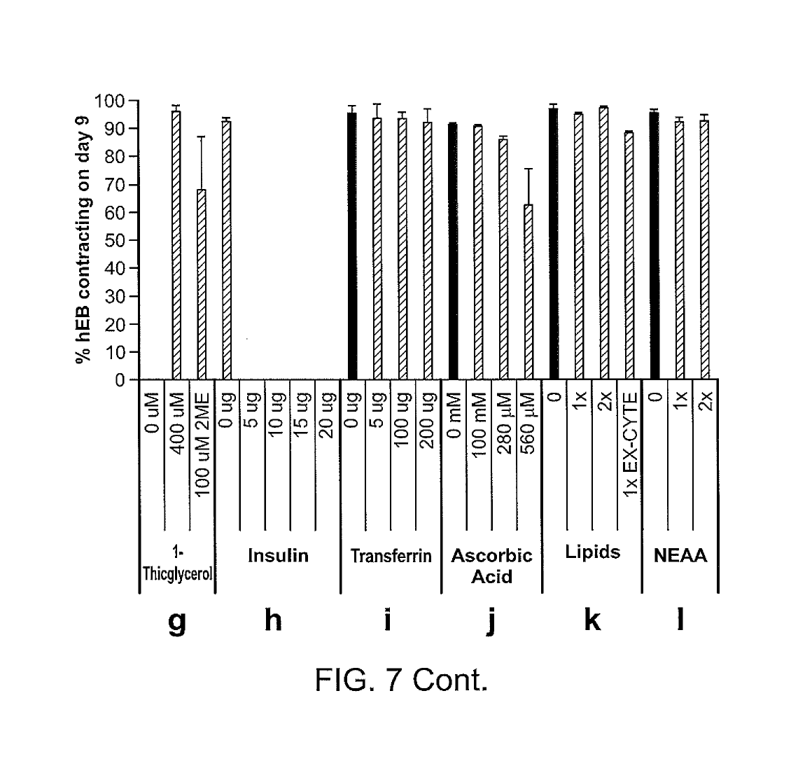

Once H9 hEB formation and mesodermal induction was reproducibly maximized, the third phase of differentiation: cardiac specification was optimized. Initially, a common cardiac differentiation medium (DMEM+20% FBS) was used for this phase (FIG. 1B). Supplementation with 20% FBS was important for efficient cardiac differentiation (FIG. 7A). The supplier of FBS did not effect cardiac differentiation, and that FBS could be substituted with 20% human serum with no reduction in efficiency (FIG. 7B). As with phase 2, RPMI 1640 was the most suitable basal media for this phase (FIG. 7E). Furthermore, supplementation with either BMP4 or FGF2 did not further enhance this phase (FIGS. 7C,D). Finally, the supplementation of the d2-d4 step with any level of insulin completely abrogated cardiac specification (FIG. 7H).

Phase 4 (d4+): heb Adherence and Chemically Defined Media Enhances Final Cardiomyocyte Differentiation

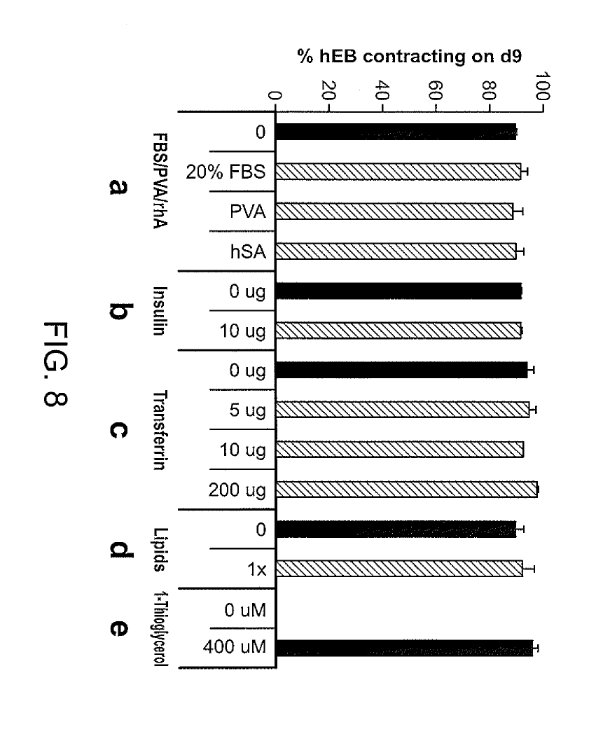

Once formulation of the optimal cardiac specification media was completed, the final steps of cardiomyocyte differentiation and maintenance were optimized. Adherence onto tissue culture treated plates on d4, although not essential, enhanced subsequent cardiomyocyte development and also eased the visualization and scoring of contraction (FIG. 1 and FIG. 6D). Unlike the third phase, the media formulation for this fourth phase was less stringent and independent of serum factors (FIG. 8A). Moreover, once contraction had begun, hEB could be successfully maintained in a variety of media (e.g. RPMI+FBS or RPMI+PVA or simple RPMI+1TG) for at least 3 months with continuous contraction of hEB. Of note, transfer of hEB to RPMI+PVA or RPMI+1TG rather than RPMI+FBS at d4 increased the range of d0-d2 BMP4 concentrations that were successful in inducing cardiac differentiation.

### Example 2

### Physiological Oxygen Tension and Polyvinyl Alcohol (PVA) Synergize to Induce Highly Efficient Cardiac Differentiation of hiPSC

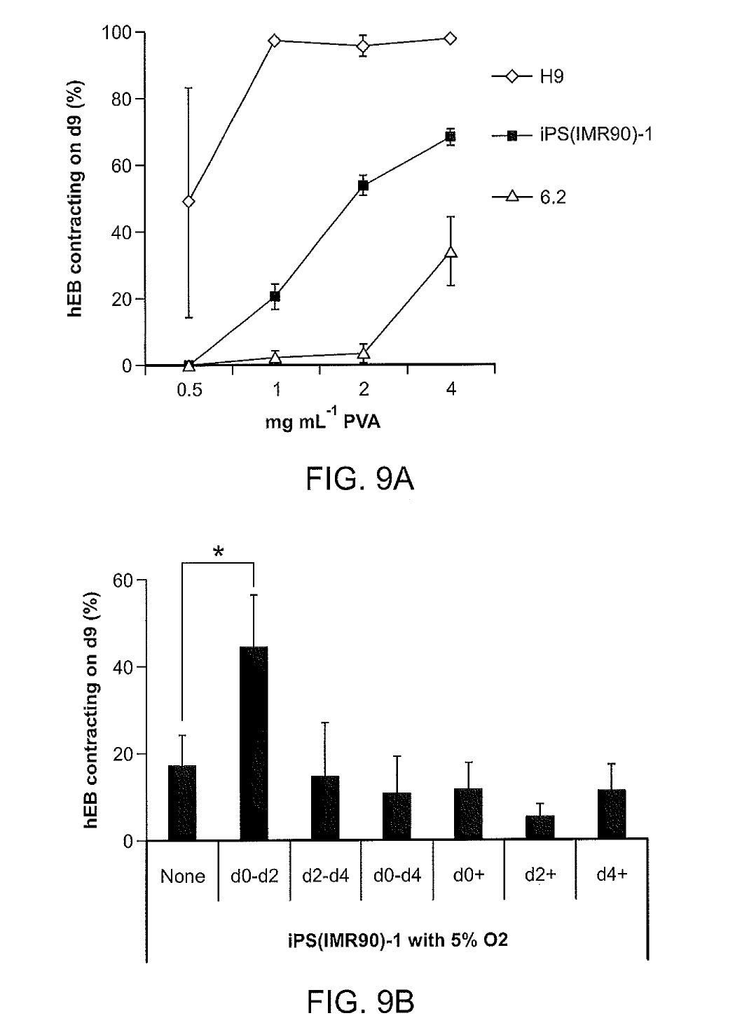

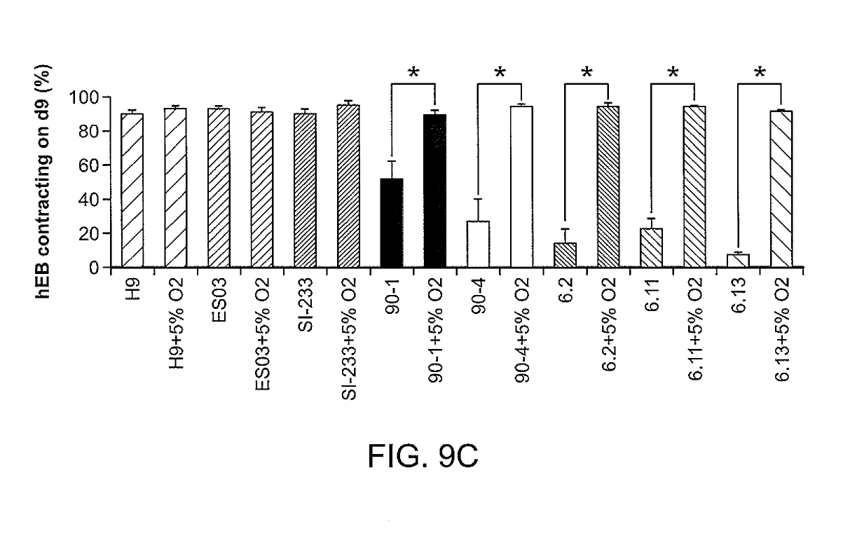

The performance of the H9 hESC-optimized cardiac differentiation protocol was assessed on several other hESC lines including HES3 (ES03) and SI-233 (SC233), and found that similarly high efficiencies of cardiac differentiation could be achieved. This system produced low levels (2.5-20.5%) of cardiac differentiation for certain hiPSC lines (FIG. 9A). Attempts to re-optimize the dose-response dependant variables from phases two and three using the hiPSC line iPS(IMR90)-1 resulted in identical optimal conditions to those found for H9, except in the case of BMP4 which was found to be most effective at 5 ng mL-1 rather than 25 ng mL-1 for H9. The main impediment to efficient hiPSC differentiation was that hiPSC-derived hEB were substantially less stable and robust (i.e. the hiPSC hEB would disintegrate during the experiment) than those formed from hESC lines. To improve this instability, the inclusion of extracellular matrix proteins (1:100 Matrigel or laminin-511 and nidogen-1) (Evseenko et al. Stem Cells Dev 18, 919-928 (2009).) during hEB formation (at d0-d2, d2-d4 or d0-d4) was tested. This completely abrogated cardiac differentiation (data not shown). However, the inclusion of increasing concentrations of the synthetic polymer polyvinyl alcohol (PVA; from 1 mg mL-1 to 4 mg mL-1) was highly effective in increasing the differentiation efficiency of iPS(IMR90)-1 hiPSC from 20.6±3.7% to 68.3±2.3%, and non-viral cord blood-derived hiPSC line 6.2 from 2.5±1.8% to 34.2±10.2% (FIG. 9A). Concentrations of PVA above 4 mg mL-1 were less effective at improving cardiac differentiation. This increased PVA concentration did not affect hESC experiments that consistently differentiated at high (>91.4%) efficiencies at 1, 2, or 4 mg mL-1 (FIG. 9A). The effects of physiological oxygen tensions on differentiation efficiency was tested by subjecting differentiation cultures to 5% O₂ at timed intervals (e.g. during d0-d2, d2-d4, d4 onwards, or combinations thereof). These experiments revealed that 5% O₂ between d0-d2 significantly (p<0.028) enhanced the differentiation of hiPSC lines tested (FIG. 9B), but had little effect on efficiency of hESC differentiation (FIG. 9C). Remarkably, the combined use of higher concentrations of PVA and timed exposure to physiological oxygen tensions significantly (p<0.04) enhanced cardiac differentiation and allowed each of the five hiPSC lines tested to achieve cardiac differentiation with a 94.7±2.4% average efficiency (FIG. 9C).

### Example 3

### hPSC-Derived Cardiomyocytes Display Functional Cardiac Properties Including Electrophysiological Profiles and Drug Response

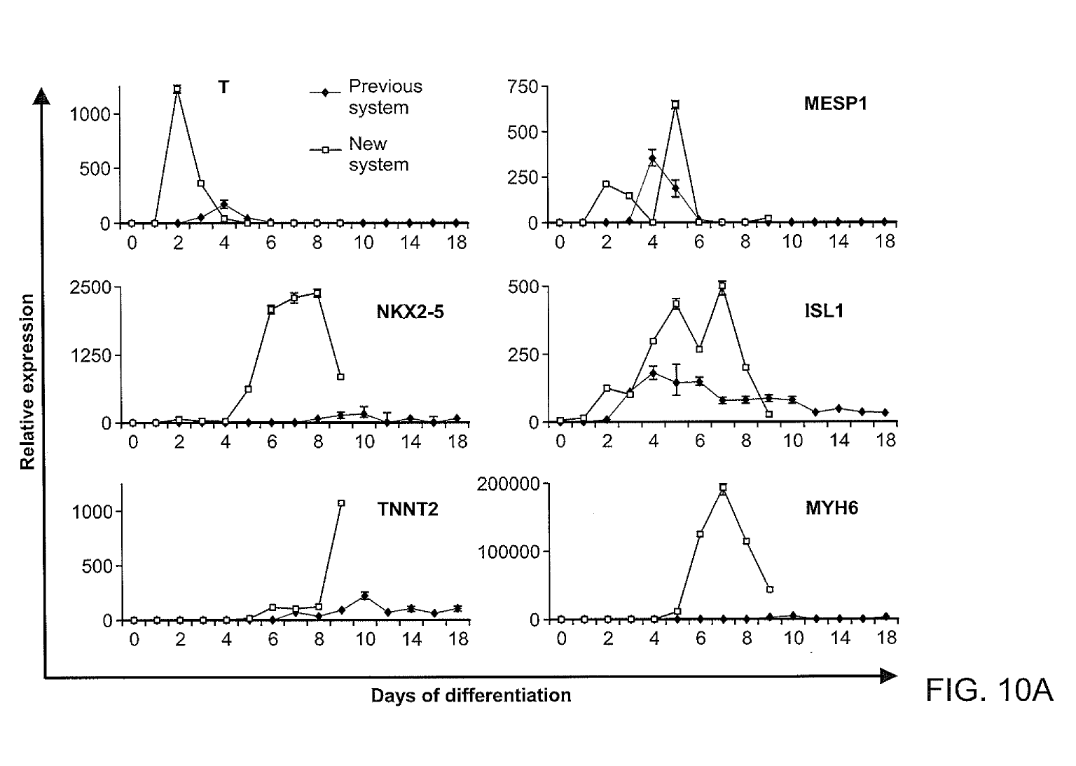

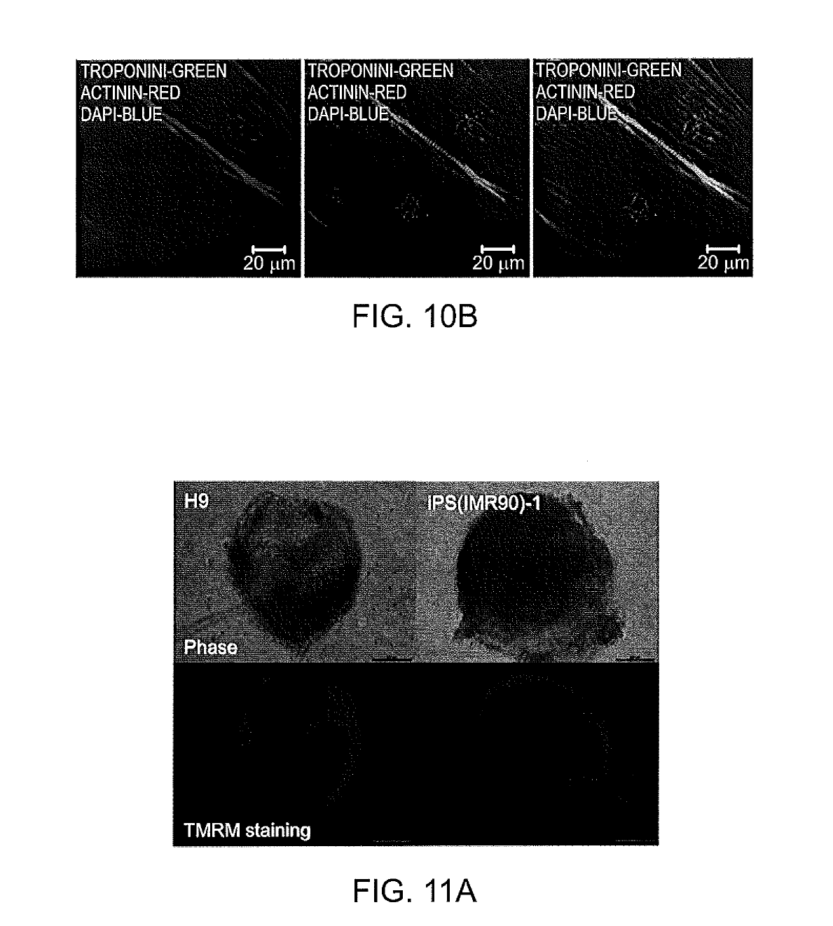

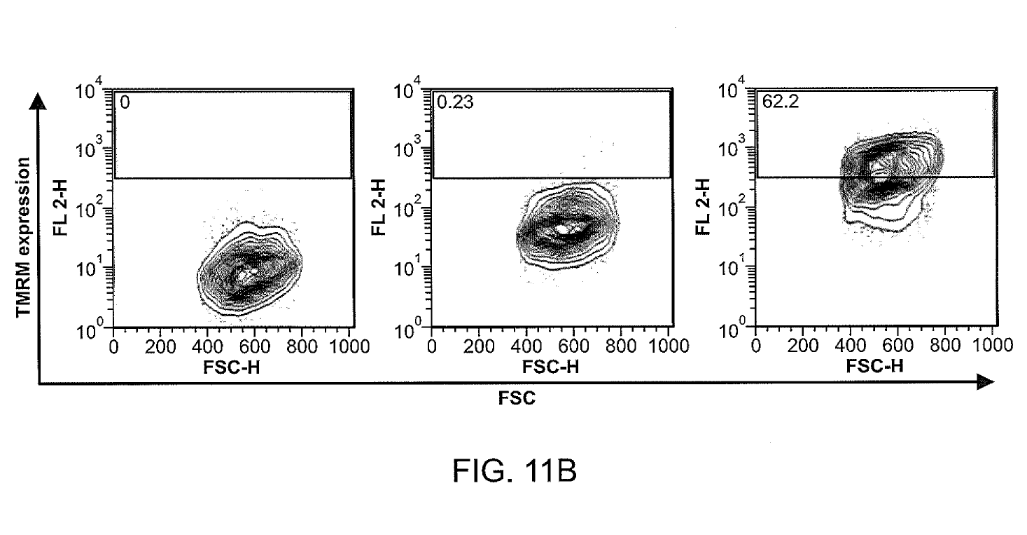

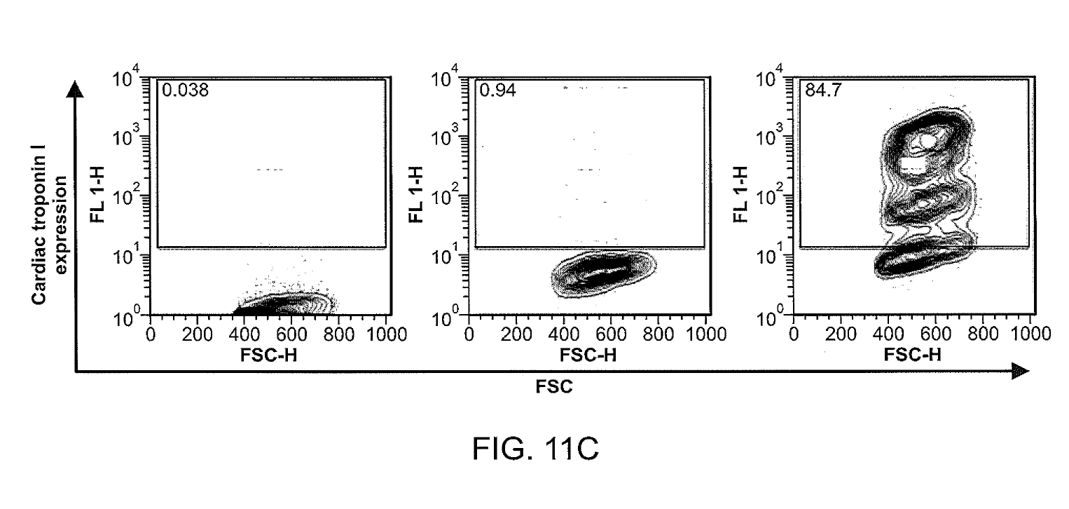

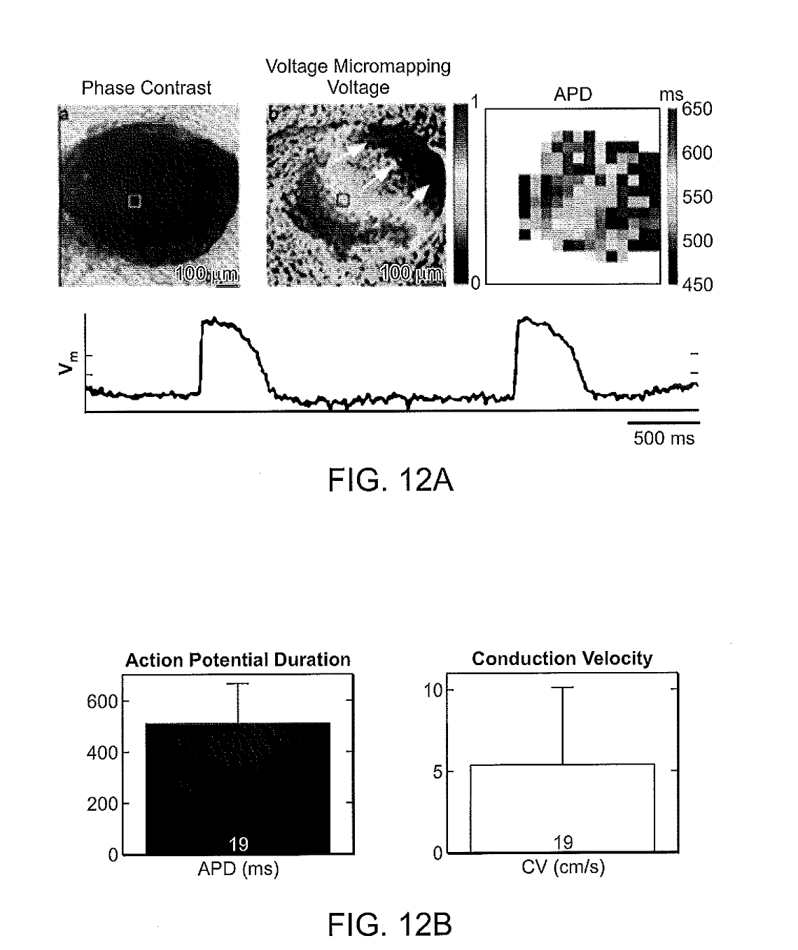

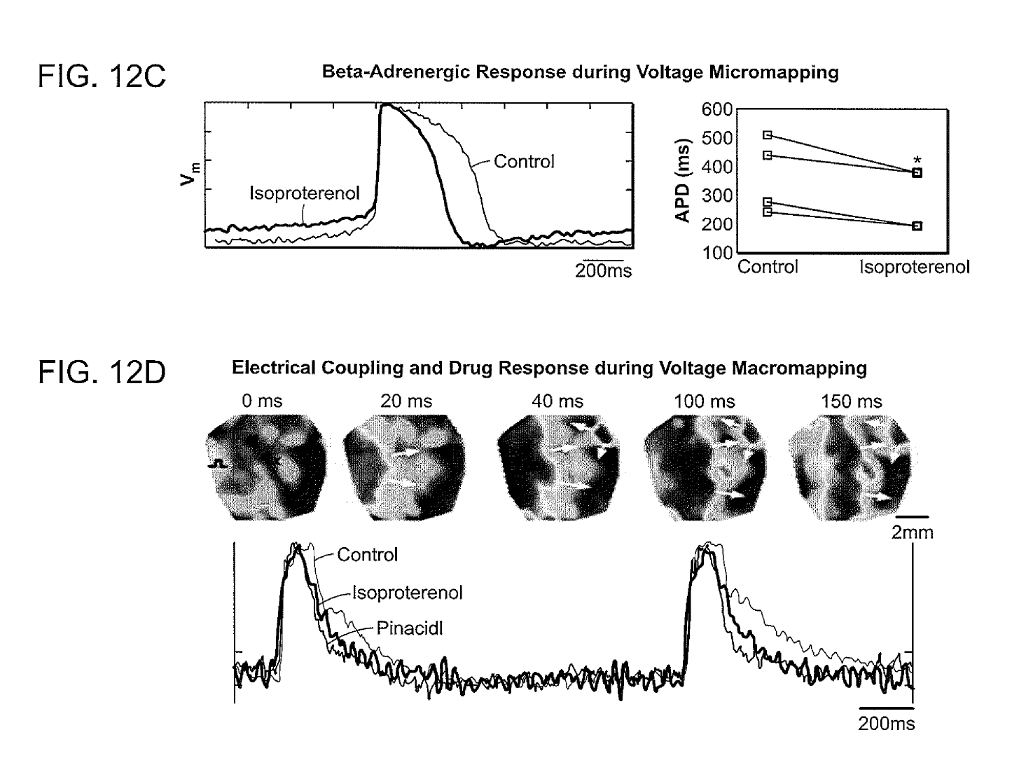

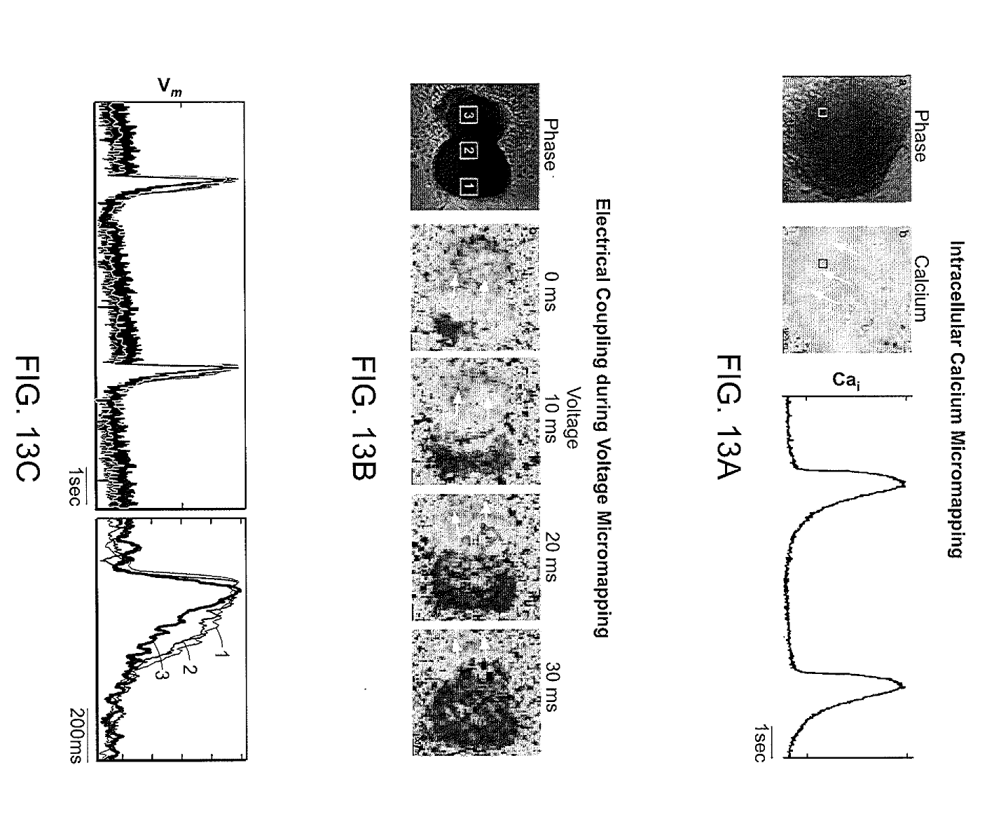

To determine whether hEB differentiated using this optimized method progress through the normal stages of cardiac lineage gene expression, and determine whether spontaneously beating cells possess normal characteristics of human cardiomyocytes, real-time quantitative RT-PCR was used to assess the kinetics of the expression of cardiac differentiation landmarks (FIG. 10A). The data collected demonstrated that using this optimized differentiation system the relative peak in mesodermal gene expression (assayed by expression of T (Brachyury) and MESP1) was substantially increased (e.g. 2-6-fold) whilst first occurrence of expression was shortened to from 4 to 2 days relative to known differentiation protocols. Expression of cardiac progenitor markers (NKX2-5 and ISL1), and terminal cardiac markers (TNNT2 and MYH6) was also substantially enhanced (e.g. 5-2500-fold) (FIG. 10A). Cardiac markers and structural protein expression was assessed using immunofluorescence staining. Cardiomyocytes derived with this method displayed striated sarcomere formation that stained strongly for sarcomeric α-actinin (ACTN2) and cardiac troponin I (TNNI3) (FIG. 10B). The presence of gap junction formation was also demonstrated by expression of CX43 (GJA1). The percentage of cardiomyocytes per hEB was also quantitated using the mitochondrial dye TMRM18 (FIGS. 11A,B), and flow cytometry for cardiac troponin I (FIG. 11C), and revealed that contracting hEB consisted predominantly (˜70-85%) of cardiomyocytes. To assess quality of hPSC-derived cardiomyocytes that were generated using this method and suitability for future applications such as cardiotoxicity testing, the electrophysiological properties were assessed via optical mapping. hEB were either mechanically dissected and plated onto fibronectin-coated glass coverslips for micromapping, or approximately 200 hEB were dissociated into single cells and plated in a 10 mm diameter area as a confluent monolayer for macromapping. hEB and monolayers were then stained with either voltage- or calcium-sensitive dye and optically mapped to visualize spontaneous activity and response to electrical field stimulation (FIG. 12A). Replicates of voltage micromapping experiments (n=19) demonstrated that electrophysiological properties, action potential duration and conduction velocity, of hEB formed using this protocol were highly reproducible (FIG. 12B). Intracellular calcium was optically mapped to demonstrate a physiological calcium transient (FIG. 13A). Functional electrical coupling between a hEB pair (FIGS. 13B,C) and within a cardiomyocyte monolayer (FIG. 12D) was demonstrated by voltage mapping. To assess cardioactive drug responsiveness, 20 μM isoproterenol or 100 μM pinacidil was added to cardiomyocyte monolayers or hEB to test for beta-adrenergic stimulation response and the presence of functional KATP channels, respectively. Both drugs produced a shortening of the action potential (FIG. 12C-D). Contracting hEB derived from the hiPSC line 6.2 were tested in the same manner and yielded similar results.

### Example 4

### Optimized Cardiac Differentiation System Using Xeno-Free and Serum-Free Reagents

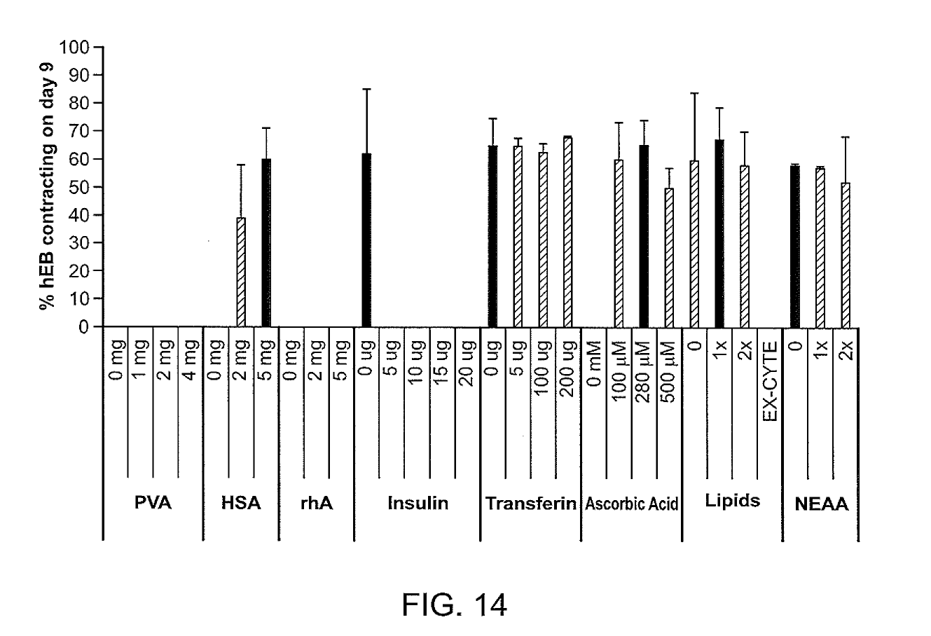

To maximize the ultimate clinical utility of the method, parallel optimization experiments that focused were conducted on the complete elimination of serum during the third phase of differentiation (d2-d4). Although a xeno-free version of this method that replaced FBS in phase 3 (d2-d4) with human serum was equally efficient, the xeno-free, chemically defined RPMI+PVA medium was not suitable for this window of differentiation (FIG. 14A). Therefore a serum-free optimal media containing human serum albumin (HSA), L-ascorbic acid, and lipids was formulated (FIGS. 14B, 14F, 14G). This formula, used from d2-d4, produced 64.8±3.3% of hEB contracting by d15 of differentiation (FIG. 3D). Additional supplementation with DKK1 and VEGFA165 7 did not further enhance differentiation.

In these studies, using systematic and rigorous optimization of culture conditions, significantly improved the cardiac differentiation of H9 hESC to an average of 94.8±1.8% efficiency. Simple modifications of employing physiological oxygen tensions and higher concentrations of PVA resulted in the development of a universal protocol that produced comparable differentiation efficiencies (average 94.7±2.4%) in three hESC and five hiPSC lines. The variation in cardiac differentiation potential among different hPSC lines cultured under similar conditions has been documented (Osafune, K. et al. Nat Biotechnol 26, 313-315 (2008); Pekkanen-Mattila, M. et al. Ann Med 41, 360-370 (2009); Burridge et al., Stem Cells 25, 929-938 (2007). Although robust cardiac differentiation in select hPSC lines has been reported (Laflamme, et al. Nat Biotechnol 25: 1015-1024 (2007); Yang et al., Nature 453: 524-528 (2008); Takei et al., Am J Physiol Heart Circ Physiol 296: H1793-1803 (2009); Gai, et al. Cell Biol Int 33: 1184-1193 (2009)), highly efficient cardiac differentiation of multiple independently derived hESC and hiPSC using a single technique has not been possible. One reason for this difficulty may be that significant variability exists in the innate response to cardiac inductive factors among different hESC lines (Kehat, I. et al., J Clin Invest 108, 407-414 (2001); Zhang et al. Circ Res 104, e30-41 (2009); Burridge et al., Stem Cells 25, 929-938 (2007). Previous cardiac differentiation systems may simply leverage the innate cardiac differentiation propensity of specific hESC lines and therefore not be suitable for other hESC lines with differing propensities. Such cardiac differentiation systems may be even less effective for hiPSC differentiation, as these cell types have been demonstrated to possess wider variation in gene expression (Chin et al. Cell Stem Cell 5, 111-123 (2009)). In developing a universal cardiac differentiation system, the strengths of multiple protocols were evaluated and tested in a multivariate fashion. Although a monolayer differentiation based technique was initially favored due to its simplicity and potential reproducibility, this protocol was ineffective for H9 hESC. Monolayer based differentiation systems have been demonstrated to be less responsive to cardiac inductive factors than the hEB format (Tran, Stem Cells 27, 1869-1878 (2009)). hEB could be formed from feeder-free single-cell hESC using forced aggregation in chemically defined media (CDM) (Wiles and Johansson, Exp Cell Res 247, 241-248 (1999)). The use of CDM enhanced the effectiveness of recombinant growth factors due to the exclusion of FBS or BSA24. A large number of mesodermal morphogens were tested for cardiogenic potential from the NODAL, BMP4, and WNT signaling cascades. However, only BMP4 was found necessary and sufficient for highly efficient cardiac differentiation. Indeed, BMP4 is a known potent mesoderm inducer in hESC25, with a brief temporal window of effectiveness of mesendoderm induction (d1-d2) (Jackson et al., PLoS One 5, e10706 (2010). Furthermore, since BMP4 and FGF2 are known to synergize for mesoderm induction (Zhang, Blood 111, 1933-1941 (2008)), we tested the effects of a BMP4/FGF2 combination during phase 2 (d0-d2) of this system and found that this combination further enhanced the differentiation efficiency. Either 20% FBS or human serum was important for phase 3 (d2-d4) of this system. The serum- and xeno-free variant of this protocol, replacing FBS/human serum with human serum albumin, lipids and L-ascorbic acid, was not as effective, suggesting that additional serum factors are important at this stage.

Once a system for highly efficient cardiac differentiation in multiple hESC lines was established, hiPSC cardiac differentiation efficiency was substantially poorer than that of hESC, as reported in other differentiation systems (Zhang et al. Circ Res 104, e30-41 (2009), These results were consistent with the notion that there are important biological differences between hiPSC and hESC. Indeed, it has been demonstrated that iPSC likely retain an epigenetic memory of their somatic cell of origin. Such inherent epigenetic limitations of iPSC lineage-specific differentiation have been partially overcome by the use of chromatin-modifying drugs (e.g. the demethylation inhibitor 5-azacytidine (Gai, Cell Biol Int 33, 1184-1193 (2009); Kim, Nature 467:285-290 (2010)) or the HDAC inhibitor trichostatin A27). In contrast, superior hiPSC cardiac differentiation efficiencies were achieved in the system of the invention without need for these non-specific, toxic, and potentially mutagenic drugs. Instead, comparably high efficiencies were achieved in both hESC and hiPSC by simply enhancing the structural integrity of hEB using PVA, and employing timed exposure to physiological oxygen tensions during the differentiation protocol. PVA, a common constituent of embryo culture media, is a simple and cost-effective media additive that is used to replace the need for BSA or FBS that can also function as an adhesive. Low oxygen tensions affect a wide range of developmental processes including cardiogenesis, the stem cell niche, and modulation of NODAL, VEGF, WNT and NOTCH signaling. Low oxygen tensions have also been implicated in improving embryoid body formation, thus it is likely that low O₂ affects multiple aspects of phase 2 of the cardiac differentiation system.

In summary, this protocol produces a near-total efficiency of cardiomyocyte differentiation from a wide variety of independently derived hESC and hiPSC lines. Importantly, the contracting cells produced using this system expressed normal cardiomyocyte markers, were capable of electrically coupling, and displayed highly reproducible electrophysiological profiles. The application of a universal cardiac differentiation protocol that can now translate across multiple hPSC allows for immediate application to hESC lines not known to be amenable to cardiac differentiation, or to genetically diverse hiPSC lines created from patients with cardiac related diseases (e.g. long QT syndrome). The uniformity of electrophysiological profiles of these cells highlights the potential for translation of this methodology to future high-throughput cardiotoxicity testing and novel drug discovery assays that can be used at various stages of drug development. Thus, this methodology greatly facilitates the utility of hiPSC-based cardiac disease modeling, drug development and cardiotoxicity screening, and the future generation of clinically-safe human cardiac cells for regenerative medicine.

### Human Pluripotent Stem Cell Culture.

All tissue culture reagents were purchased from Invitrogen unless otherwise stated. MEF, hESC and hiPSC culture were maintained at 37° C., 5% CO2 and 85% relative humidity. Medium was changed every day on hESC and hiPSC cultures. Physiological oxygen tension differentiations were performed at 37° C., 5% CO2, 5% o2 and 85% relative humidity. Physiological oxygen conditions were created in a hypoxia chamber (Billups-Rothenburg) using nitrogen gas controlled by a ProOx Oxygen Controller (BioSpherix). hESC line H9 (WA09)31 and hiPSC lines iPS(IMR90)-1-DL-1 and iPS(IMR90)-4-DL-1 derived from fibroblasts using lentiviruses32 were purchased from the WiCell WISC Bank. The hESC line ES0333 was purchased from ES Cell International and hESC line SI-23334 was purchased from Stemride International. hiPSC lines 6.2, 6.11, and 6.13 were derived from CD34⁺ cord blood using a modified non-integrating episomal plasmid methodology. All hESC lines used in these studies were approved for use by the Johns Hopkins University Institutional Stem Cell Research Oversight Committee (ESCRO) (http://www.hopkinsmedicine.org/Research/iscro/JHU_ISCRO_Registry.html). All pluripotent cell lines were initially cultured as colonies on MEF (E13.5 DR4 seeded at 2×10⁴ cells cm⁻²) in 6-well plates (Greiner Bio-One) in hESC medium consisting of DMEM-F12 (with GlutaMAX-I, no HEPES), 15% Knockout Serum Replacer (KSR), 1% non-essential amino acids (NEAA), 100 μM 2-mercaptoethanol and 4 ng mL-1 FGF2 (R&D Systems) and passaged with 1 mg mL-1 collagenase IV for 5 min at 37° C. Conditioned medium was made essentially as previously described (Xu et al. Nat Biotechnol 19, 971-974 (2001)): In brief, confluent p2 MEF were irradiated (5000 cGy) and seeded at 6×10⁴ cells cm⁻² on gelatin coated flasks in MEF medium consisting of DMEM (with Glutamine), 10% fetal bovine serum (Characterized, Hyclone), 1% NEAA, 55 μM 2-mercaptoethanol. After allowing MEF to attach for 24 hours media was replaced with 0.5 mL cm-2 hESC medium. Medium was conditioned for 22-26 hours, pooled, filter sterilized and supplemented with an additional 4 ng mL-1 FGF2 and stored at −20° C. Conditioned medium was collected for 7 days. For transfer of hiPSC and hESC to monolayer culture 36 l confluent well of a 6-well plate was treated with room temperature trypsin, commercially available as TrypLE Select, for 2 minutes at 37° C. and single cells were passaged into a T25 flask (BD Biosciences) coated with a 1:400 dilution (200 μL cm⁻²) of Geltrex in 5 mL of conditioned medium. Confluent cultures were passaged every 3 days by washing with PBS then treating with room temperature TrypLE Select for 1 min at 37° C. Cells were counted with a Countess Automated Cell Counter and seeded at 1.25×10⁶ cells per T25 flask. All hPSC lines commonly grew from 1.25×10⁶ to 5-6×10⁶ in 3 days.

### Cardiac Differentiation.

For forced aggregation hEB differentiations, confluent hESC or hiPSC which had been grown on a commercially available extracellular matrix, Geltrex (Invitrogen) as monolayers for 3 to 13 passages were passaged with TrypLE Select and seeded at 2.5×10⁶ per T25 flask. After 24 hours growth, cells were washed with PBS and treated with room temperature TrypLE Select for 1 minute at 37° C. Cells were seeded at 5000 cells per well in 96-well V-bottom uncoated plates (249952, NUNC) in 100 μL per well RPMI+PVA medium consisting of RPMI Media 1640 (with L-Glutamine), 4 mg mL-1 polyvinyl alcohol (P8136, Sigma, dissolved in RPMI at 4° C. for at least 72 hours, mixing by inversion every day), 1% chemically defined lipid concentrate, 10 μg mL-1 recombinant human insulin (Sigma), 400 μM 1-thioglycerol (Sigma), 25 ng mL-1 BMP4 and 5 ng mL-1 FGF2 (both from R&D systems), 1 μM Y-27632 (Calbiochem). This medium is not stable and was made fresh for each experiment. After 48 hours (on day 2) medium was aspirated with a Costar 8-channel aspirator (Corning) and replaced with RPMI+FBS medium consisting RPMI Media 1640, 20% characterized FBS (Hyclone), 400 μM 1-thioglycerol. On day 4 media was changed to RPMI+PVA without growth factors and hEB were transferred to 96-well U-bottom tissue culture treated plates (650180, Greiner Bio-One). Media was changed on d7 and every 3 days afterwards. hEB were visually assessed for contraction on d9 using a Nikon Eclipse Ti inverted tissue culture microscope with a TOKAI-HIT heated stage (Nikon). Images were captured using NIS-Elements (Nikon). Other factors that were tested include: Germcell human serum (Gemini), Matrigel (BD Biosciences), NODAL, activin A, TDGF1, BMP2, BMP6, TGFB, IGF1, IGF2, Nidogen (R&D systems), 96-well U-bottom uncoated plates (NUNC), 96-well F-bottom tissue culture plates (Greiner Bio-One), ITS-X, ITS-G, N2 supplement, B27 supplement, bovine transferrin, non-essential amino acids, DMEM, IMDM, F12, KO-DMEM, StemPro-34 (all from Invitrogen), X-VIVO 10 (Lonza), BSA, L-ascorbic acid, Stemline II (Sigma), mTeSR1, SFEM (StemCell Technologies), mouse WNT3A, EX-CYTE (Millipore). For traditional cardiac differentiations using FBS, undirected differentiation was performed as previously described (Kehat et al. J Clin Invest 108, 407-414 (2001)): briefly, confluent H9 hESC grown as colonies on MEF were treated with collagenase IV for 5 minutes at 37° C. then washed from the plate using a 5 mL pipette. Cell clusters were then transferred to Petri dishes in 20% FBS medium for 7 days. hEB were then transferred to gelatin coated tissue culture plates. Media was changed every 3 days.

### Statistical Design.

One single replicate consists of one 96-well plate. Repeat replicates were performed 1-4 months apart using a different vial of cells. Each experiment was repeated at least 3 times representing at least 288 hEB. Total number of hEB assessed in this work exceeds 80,000. Wells in which no hEB was detected due to pipeting error were excluded and accounted for approximately 1-5% of wells. P-values were established using unpaired two-tailed Student's t-test.

### Quantitative Real-Time RT-PCR.

hEB were collected, washed in PBS and flash frozen at −80° C. in RLT buffer (QIAGEN). RNA was extracted using an RNeasy kit (QIAGEN), analyzed on a NanoDrop ND1000 (ThermoScientific) and cDNA synthesis performed using a High Capacity RNA-cDNA kit (Applied Biosystems). Real-time quantitative RTPCR was performed using Universal PCR Master Mix (Applied Biosystems) and Taqman Assay-on-Demand Gene Expression Assays (Applied Biosystems) on an Applied Biosystems 7900HT. The list of Assay-on-Demand Gene Expression Assays (RT-PCR primer pairs) used in these studies is included in Table 2.

TABLE 2
Real Time Quantitative RT-PCR Primers

| Gene Symbol | Assay ID | Gene Name | NCBI Gene Reference |
|---|---|---|---|
| POU5F1 | Hs00999632_g1 | POU class 5 homeobox 1 | NM_203289.3 |
| NANOG | Hs02387400_g1 | Nanog homeobox | NM_024865.2 |
| T | Hs00610080_m1 | T, brachyury homolog (mouse) | MM_003181.2 |
| MESP1 | Hs00251489_m1 | mesoderm posterior 1 | NM_018670.3 |
| | | homolog (mouse) | |
| RBM24 | Hs00290607_m1 | RNA binding motif protein 24 | NM_153020.2 |
| KIT | Hs00174029_m1 | v-kit Hardy-Zuckerman 4 | NM_001093772.1 |
| | | feline sarcoma viral | |
| | | oncogene homolog | |
| KDR | Hs00911700_m1 | kinase insert domain | NM_002253.2 |
| | | receptor (a type III receptor | |
| | | tyrosine kinase) | |
| ISL1 | Hs00158126_m1 | ISL LIM homeobox 1 | NM_002202.2 |
| NKX2 5 | Hs00231763_m1 | NK2 tanscription factor | NM_004387.3 |
| | | related, locus 5 (Drosophila) | |
| TNNT2 | Hs00165960_m1 | toponin T type 2 (cardiac) | NM_001001430.1 |
| MYH6 | Hs00411887_m1 | myosin, heavy chain 6, | D00943.1 |
| | | cardiac muscle, alpha | |
| 18S | Hs99999901_s1 | Eukaryotic 18S rRNA | X03205.1 |

### Intracytoplasmic and TMRM Mitochondrial Dye FACS Analysis.

Whole cardiomyocyte hEB clusters were plated onto fibronectin-coated glass coverslips and given 5 days to attach. Cells were washed with PBS and fixed in cold 3.7% paraformaldehyde (PFA) for 10-15 minutes, washed in PBS and permeabilized with 0.2% Triton X (Sigma) in Tris buffered saline (TBS). Cells were then incubated with 10% Goat Serum (Sigma) in PBS for 1 hour at room temperature.

### Antibodies

Sarcomeric Alpha Actinin antibody (ab9465, Abcam) and Anti-troponin I, Cardiac (cTnI) (T8665-13F, US Biological) were diluted 1:200 in antibody diluent (IHC World) and incubated overnight at 4° C. Cells were washed 3 times in TBS-T then secondary antibodies: 568 Goat anti-mouse IgG, 488 Goat anti-mouse IgG2b, (Invitrogen) were diluted 1:200 antibody diluent and incubated for 45 min at RT in the dark. Cells were then washed 3 times in TBS-T and mounted with ProLong Gold with DAPI (Invitrogen) onto Superfrost Plus (VWR) slides and imaged with a Confocal Microscope (Zeiss). For flow cytometry 1 whole plate of d9 H9 were disaggregated using TrypLE and single cells were fixed and permeablized using Fix & Perm Kit (Invitrogen) and stained using the above troponin I antibody. For TMRM dye staining, whole d9 H9 hEB were stained with 10 nM TMRM (tetramethylrhodamine, methyl ester, perchlorate, Molecular Probes/Invitrogen) in RPMI at 37° C. for exactly 30 min. Media was then replaced with RPMI only for 30 min to remove any unbound dye. hEB then were manually removed from 96-well plates and disaggregated into single cells using TrypLE for 5 min, followed by manual trituration. Single cells were analyzed by flow cytometry using a FACSCaliber instrument (Beckton Dickinson). Data was analyzed using FlowJo flow cytometry analysis software (Tree Star).

### Cardiomyocyte Electrophysiology.

For optical micromapping (Weinberg et al., Methods Mol Biol 660, 215-237 (2010), contracting hEB were mechanically dissected, plated on fibronectin-coated glass coverslips and given at least 5 days to attach. hEB were then stained with either 10 μM Rhod-2-AM calcium dye for 20 minutes or 10 μM di-4-ANEPPS voltage dye for 5 minutes. After several rinses with Tyrode's solution (135 mM NaCl, 5.4 mM KCl, 1.8 mM CaCl2, 1 mM MgCl2, 0.33 mM NaH2PO4, 5 mM HEPES, and 5 mM glucose), hEB were incubated with 30 μM blebbistatin for 15 minutes to inhibit excitation-contraction coupling and subsequently prevent signal distortion due to motion artifact. The absence of hEB contraction was confirmed visually. hEB were then excited at 530 nm to visualize spontaneous activity and response to electrical field stimulation. Imaging of transmembrane potential (Vm) or intracellular calcium (Cai) was performed using an Andor iXon+860 electron multiplying charged coupled device (EMCCD) camera (128×128 pixels) at 490 Hz sampling rate. At 6× magnification, the field of view is ˜520 μm×520 μm, resulting in a spatial resolution of ˜4 Micromapping experiments were performed at room temperature. Macromapping of hESC-CM monolayers was performed using contact fluorescent imaging, in which maps of Vm were recorded by placing the monolayer directly on top of a bundle of 253 optical fibers 1 mm in diameter, arranged in a tightly packed, 17-mmdiameter hexagonal array. The cell monolayers were stained with 10 μM di-4-ANEPPS, and continually superfused with Tyrode's solution. The monolayer was excited by an array of high-power green LEDs placed directly above the experimental chamber. The fluorescent dye signal was relayed by the optical fiber bundle to an array of photodetectors and amplifiers, digitized at a 1 kHz sampling rate, and processed by custom written software. Macromapping experiments were performed at 36° C. In drug response experiments, drugs were added for 15 min before subsequent recordings. To analyze data, the individual recorded signals recorded were spatially filtered using a 5×5 box filter, temporally filtered using a 10 point median filter, baseline-corrected by subtraction of a fitted 3rd order polynomial, and range-normalized. The activation time at each recording site was computed as the time of the maximum first derivative of the action potential (dVm/dtmax) or calcium transient upstroke (dCa/dtmax). Repolarization time was computed as the 80% recovery time from the peak amplitude, and action potential duration (APD) was computed from the difference of repolarization and activation times. APD maps were computed by first spatially binning voltage data to 16×16 pixels and measuring APD at each pixel. Uniformity of APD was assessed by the coefficient of variation. For each hEB, the coefficient of variation was determined from the mean APD (over all pixels in the APD map), divided by the standard deviation. Conduction velocity was computed by taking the distance of a path perpendicular to the direction of propagation, and dividing by the difference of activation times at the path endpoints. At least 3 paths were chosen for each measurement. The conduction velocity coefficient of variation was determined from the mean conduction velocity (over all measured paths), divided by the standard deviation. Conduction velocity was computed by taking the distance of a path perpendicular to the direction of propagation, and dividing by the difference of activation times at the path endpoints. At least 3 paths were chosen for each measurement.

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. A method for generating a differentiated mesodermal cell, the method comprising
a) culturing a human stem cell as a monolayer on a proteinaceous cell culture matrix that supports cell adhesion;
b) promoting the aggregation of the cells by culturing the cells in media comprising poly(vinyl alcohol), BMP4, and FGF2;
c) maintaining the cells in culture for another forty-eight hours under conditions that support mesodermal lineage specification; and
d) maintaining the cells for six additional days under conditions that promote human embryonic body maturation, thereby generating a differentiated mesodermal cell.

2. The method of claim 1, wherein step b further comprises plating cells from step a at about 5000 cells per well of a 96-well plate in culture media comprising 25 ng ml⁻¹ BMP4, 5 ng ml⁻¹ FGF2 and cell culture media comprising at least 4% PVA.

3. The method of claim 1, wherein step b further comprises centrifuging the culture to force the cells to aggregate.

4. The method of claim 1, wherein the conditions of step c comprise culturing the cells in media comprising FBS or a FBS-substitute.

5. The method of claim 1, wherein the conditions of step d comprise culturing the cells in media comprising FBS or a FBS-substitute.

6. The method of claim 1, wherein the conditions of step d comprise culturing the cells in media comprising 4% PVA.

7. The method of claim 6, wherein the stem cell is selected from the group consisting of induced pluripotent stem cell, human embryonic stem cell, mesodermal stem cell, and other mesodermal stem cell.

8. The method of claim 6, wherein the stem cell is a pluripotent stem cell.

9. The method of claim 8, wherein the stem cell is an induced pluripotent stem cell.

10. The method of claim 9, wherein an induced pluripotent stem cell is derived from a somatic cell.

11. The method of claim 10, wherein the somatic cell is selected from the group consisting of keratinocyte, epidermal cell, fibroblast, and their progenitor cells.

12. The method of claim 1, wherein the human embryonic stem cell or human induced pluripotent stem cell of step a is maintained in a culture comprising medium conditioned on irradiated mouse embryonic fibroblasts seeded at 6×10⁴ cells/cm² for 22-26 hours.

13. The method of claim 1 wherein the media for the culture of pluripotent cells as a monolayer is a commercially available media selected from the group consisting of mTeSR1, TeSR2 (Stem Cell Technologies), Nutristem (Stemgent), StemPro hESC SFM (Invitrogen) or media supplemented with Xeno-Free Knockout Serum Replacement and Xeno-Free Growth Factor Cocktail (Invitrogen).

14. The method of claim 1, wherein the conditioned media of claim 12 further comprises at least about 4 ng/ml FGF2.

15. The method of claim 1, wherein the medium is RPMI-PVA medium comprising 2 mM L-glutamine, 4 mg/mL PVA, 1× Chemically Defined Lipid Concentrate, 400 mM 1-thioglycerol, 10 ug/mL insulin.

16. A method for generating a differentiated mesodermal cell, the method comprising
a) culturing a human stem cell or human induced pluripotent stem cell in medium conditioned on irradiated mouse embryonic fibroblasts seeded at 6×10⁴ cells/cm² for 22-26 hour, wherein the conditioned media comprises at least about 4 ng/ml FGF2;
b) culturing a human stem cell or human induced pluripotent stem cell of step a as a monolayer on a proteinaceous cell culture matrix that supports cell adhesion;
c) plating cells from step b at about 5000 cells per well of a 96-well plate in culture media comprising 25 ng ml⁻¹ BMP4, 5 ng ml⁻¹ FGF2 and cell culture media comprising at least 0.1% PVA and centrifuging the culture to force the cells to aggregate;
d) maintaining the cells in culture for another forty-eight hours in media comprising media comprising FBS or a FBS-substitute, 25 ng/ml BMP4 and 5 ng ml⁻¹ FGF2, thereby promoting mesodermal lineage specification; and
e) maintaining the cells for six additional days in media comprising 4% PVA or media comprising FBS or an FBS-substitute thereby generating a differentiated mesodermal cell.

17. The method of claim 16, wherein the media of step c is RPMI-BSA-PVA medium that comprises or consists essentially of RPMI with 2 mM L-glutamine, 0.5 mg/mL PVA, 1 mg/mL BSA, 1× Chemically Defined Lipid Concentrate (commercially available from Invitrogen), 400 mM 1-thioglycerol, ITS-X (consisting of 0.0067 ug/mL sodium selenite, 10 ug/mL insulin, 5.5 ug/mL transferrin and 2 ug/mL ethanoamine).

18. The method of claim 1, further comprising detecting and isolating hEBs that beat.

19. The method of claim 1, wherein the method further comprises identifying an differentiated mesodermal phenotype by detecting an increase in a mesodermal marker, mesodermal morphology, or mesodermal function that is not detectably expressed or expressed only nominally in a corresponding control cell.

20. The method of claim 1, wherein the differentiated mesodermal cell expresses one or more mesodermal markers selected from the group consisting of T, MIXL1, GSC, EOMES and MESP1.

21. The method of claim 1, wherein the method generates about 75%, 85%, 90% or 85% differentiated mesodermal cells after about 9 days.

22. A differentiated mesodermal cell generated according to the method of claim 1.

23. The differentiated mesodermal cell of claim 22, wherein said cell is a cardiac cell.

24. A culture system comprising one or more containers of media, wherein
a first cell culture media that promotes mesodermal lineage specification, the media comprising FBS or a FBS-substitute, about 25 ng/ml BMP4 and about 5 ng ml⁻¹ FGF2;
a second cell culture media that promotes cell aggregation comprising about 25 ng ml⁻¹ BMP4, about 5 ng ml⁻¹ FGF2 and about 4% PVA;
a third cell culture media for maintaining human embryonic stem cells or human induced pluripotent cells in culture, wherein the medium is conditioned on irradiated mouse embryonic fibroblasts seeded at about 6×10⁴ cells/cm² for about 22-26 hour, and wherein the conditioned media comprises at least about 4 ng/ml FGF2; and
directions for the use of the culture system to promote mesodermal cell proliferation according to claim 1.

25. The culture system of claim 24, further comprising culture flasks or culture plates.

26. A method of ameliorating cell or tissue loss in a subject in need thereof, the method comprising delivering to the subject an effective amount of a cell generated according to the method of claim 1.

27. The method of claim 26, wherein the cell or tissue loss or damage is associated with a condition selected from the group consisting of myocardial infarction, heart failure, cardiomyopathy, congenital heart disease, nutritional diseases, ischemic or non-ischaemic cardiomyopathy, hypertensive cardiomyopathy, valvular cardiomyopathy, inflammatory cardiomyopathy, cardiomyopathy secondary to a systemic metabolic disease, alcoholic cardiomyopathy, diabetic cardiomyopathy, and restrictive cardiomyopathy.

28. A method of treating a damaged cardiac tissue in a subject in need thereof, the method comprising delivering to the subject an effective amount of a cardiac myocyte generated according to the method of claim 1

29. A pharmaceutical composition comprising a differentiated mesodermal cell generated according to the method of claim 1 in a pharmaceutically acceptable excipient.

30. A kit comprising the culture system of claim 24 and instructions for generating a differentiated mesodermal cell in accordance with claim 1.

31. A kit comprising a differentiated mesodermal cell obtained according to claim 1, and instructions for engraftment of the differentiated mesodermal cell in a subject.

32. A method for drug screening, the method comprising contacting a cardiac cell of claim 23 with an agent and detecting an alteration in the survival or biological activity of the cell.

33. The method of claim 32, wherein the method detects an increase or decrease in cell death.

34. The method of claim 32, wherein the method detects an increase or decrease in biological activity.
